SEQUENCE LISTING (22 sequences)

SEQ ID NO: 1 - protein, 30 aa, synthetic construct
  SITE 1: note = N-3-(imidazol-4-yl)-propionyl-tryptophan
  SITE 30: note = N{Epsilon}-[2-[2-[2-[[2-[2-[2-[[(4S)-4-carboxy-4-(1 7-carboxyheptadecanoylamino)butanoyl]amino]ethoxy]ethoxy]a cetyl]amino]ethoxy]ethoxy]acetyl]-lysine
WEGTFTSDVSSYLEEQAAREFIAWLVRGRK

SEQ ID NO: 2 - protein, 30 aa, synthetic construct
  SITE 1: note = N-3-(imidazol-4-yl)-propionyl-glycine
  SITE 30: note = N{Epsilon}-[2-[2-[2-[[2-[2-[2-[[(4S)-4-carboxy-4-(1 7-carboxyheptadecanoylamino)butanoyl]amino]ethoxy]ethoxy]a cetyl]amino]ethoxy]ethoxy]acetyl]-lysine
GEGTFTSDVSSYLEEQAAREFIAWLVRGRK

SEQ ID NO: 3 - protein, 30 aa, synthetic construct
  SITE 30: note = N{Epsilon}-[2-[2-[2-[[2-[2-[2-[[(4S)-4-carboxy-4-(1 7-carboxyheptadecanoylamino)butanoyl]amino]ethoxy]ethoxy]a cetyl]amino]ethoxy]ethoxy]acetyl]-lysine
  SITE 1: note = N-3-(imidazol-4-yl)-propionyl-Aib; note = Aib
XEGTFTSDVSSYLEEQAAREFIAWLVRGRK

SEQ ID NO: 4 - protein, 30 aa, synthetic construct
  SITE 1: note = N-3-(imidazol-4-yl)-propionyl-alanine
  SITE 30: note = N{Epsilon}-[2-[2-[2-[[2-[2-[2-[[(4S)-4-carboxy-4-(1 7-carboxyheptadecanoylamino)butanoyl]amino]ethoxy]ethoxy]a cetyl]amino]ethoxy]ethoxy]acetyl]-lysine
AEGTFTSDVSSYLEEQAAREFIAWLVRGRK

SEQ ID NO: 5 - protein, 31 aa, synthetic construct
  SITE 31: note = N{Epsilon}-[2-[2-[2-[[2-[2-[2-[[(4S)-4-carboxy-4-(1 7-carboxyheptadecanoylamino)butanoyl]amino]ethoxy]ethoxy]a cetyl]amino]ethoxy]ethoxy]acetyl]-lysine
HWEGTFTSDVSSYLEEQAAREFIAWLVRGRK

SEQ ID NO: 6 - protein, 31 aa, synthetic construct
  SITE 31: note = N{Epsilon}-[2-[2-[2-[[2-[2-[2-[[(4S)-4-carboxy-4-(1 7-carboxyheptadecanoylamino)butanoyl]amino]ethoxy]ethoxy]a cetyl]amino]ethoxy]ethoxy]acetyl]-lysine
HGEGTFTSDVSSYLEEQAAREFIAWLVRGRK

SEQ ID NO: 7 - protein, 31 aa, synthetic construct
  SITE 2: note = Aib
  SITE 31: note = N{Epsilon}-[2-[2-[2-[[2-[2-[2-[[(4S)-4-carboxy-4-(1 7-carboxyheptadecanoylamino)butanoyl]amino]ethoxy]ethoxy]a cetyl]amino]ethoxy]ethoxy]acetyl]-lysine
HXEGTFTSDVSSYLEEQAAREFIAWLVRGRK

SEQ ID NO: 8 - protein, 31 aa, synthetic construct
  SITE 31: note = N{Epsilon}-[2-[2-[2-[[2-[2-[2-[[(4S)-4-carboxy-4-(1 7-carboxyheptadecanoylamino)butanoyl]amino]ethoxy]ethoxy]a cetyl]amino]ethoxy]ethoxy]acetyl]-lysine
HAEGTFTSDVSSYLEEQAAREFIAWLVRGRK

SEQ ID NO: 9 - protein, 30 aa, synthetic construct
  SITE 1: note = N-3-(imidazol-4-yl)-propionyl-tryptophan
WEGTFTSDVSSYLEEQAAREFIAWLVRGRG

SEQ ID NO: 10 - protein, 31 aa, synthetic construct
  SITE 2: note = Aib
HXEGTFTSDVSSYLEGQAAKEFIAWLVRGRG

SEQ ID NO: 11 - protein, 31 aa, synthetic construct
HAEGTFTSDVSSYLEGQAAKEFIAWLVKGRG

SEQ ID NO: 12 - protein, 31 aa, synthetic construct
  SITE 2: note = Aib
  SITE 20: note = N{Epsilon}-[2-[2-[2-[[2-[2-[2-[[(4S)-4-carboxy-4-(1 7-carboxyheptadecanoylamino)butanoyl]amino]ethoxy]ethoxy]a cetyl]amino]ethoxy]ethoxy]acetyl]-lysine
HXEGTFTDSVSSYLEGQAAKEFIAWLVRGRG

SEQ ID NO: 13 - protein, 30 aa, synthetic construct
  SITE 1: note = Aib; note = N-3-(imidazol-4-yl)-propionyl-Aib
  SITE 19: note = N{Epsilon}-[2-[2-[2-[[2-[2-[2-[[(4S)-4-carboxy-4-(1 7-carboxyheptadecanoylamino)butanoyl]amino]ethoxy]ethoxy]a cetyl]amino]ethoxy]ethoxy]acetyl]-lysine
XEGTFTSDVSSYLEGQAAKEFIAWLVRGRG

SEQ ID NO: 14 - protein, 31 aa, synthetic construct
  SITE 20: note = N{Epsilon}-[2-[2-[2-[[2-[2-[2-[[(4S)-4-carboxy-4-(1 7-carboxyheptadecanoylamino)butanoyl]amino]ethoxy]ethoxy]a cetyl]amino]ethoxy]ethoxy]acetyl]-lysine
HWEGTFTSDVSSYLEGQAAKEFIAWLVRGRG

SEQ ID NO: 15 - protein, 30 aa, synthetic construct
  SITE 1: note = N-3-(imidazol-4-yl)-propionyl-tryptophan
  SITE 19: note = N{Epsilon}-[2-[2-[2-[[2-[2-[2-[[(4S)-4-carboxy-4-(1 7-carboxyheptadecanoylamino)butanoyl]amino]ethoxy]ethoxy]a cetyl]amino]ethoxy]ethoxy]acetyl]-lysine
WEGTFTSDVSSYLEGQAAKEFIAWLVRGRG

SEQ ID NO: 16 - protein, 31 aa, synthetic construct
  SITE 24: note = N{Epsilon}-[2-[2-[2-[[2-[2-[2-[[(4S)-4-carboxy-4-(1 7-carboxyheptadecanoylamino)butanoyl]amino]ethoxy]ethoxy]a cetyl]amino]ethoxy]ethoxy]acetyl]-lysine
HVEGTFTSDVSSYLEEQAAREFIKWLVRGRG

SEQ ID NO: 17 - protein, 31 aa, synthetic construct
  SITE 1: note = D-tyrosine
  MOD_RES 1: note = N-Acetyl-d-tyrosine
  SITE 2: note = Aib
  SITE 14: note = Nle
  SITE 24: note = N-epsilon-[(4S)-4-carboxy-4-[[(4S)-4-carboxy-4-[[(4 S)-4-carboxy-4-[[4-[(19-carboxynonadecanoylamino)methyl]cy clohexanecarbonyl]amino]butanoyl]-amino]butanoyl]amino]but anoyl]-lysine
  MOD_RES 31: note = C-terminal amidation
YXEGTFISDYSIAXDKIRQQDFVKWLLAQKP

SEQ ID NO: 18 - protein, 30 aa, synthetic construct; identical to SEQ ID NO: 3
  VARIANT 1: note = N-3-(imidazol-4-yl)-propionyl-tryptophan or N-3-(imidazol-4-yl)-propionyl-glycine

SEQ ID NO: 19 - protein, 30 aa, synthetic construct; identical to SEQ ID NO: 3
  VARIANT 1: note = N-3-(imidazol-4-yl)-propionyl-tryptophan or N-3-(imidazol-4-yl)-propionyl-glycine
  SITE 30: note = N{Epsilon}-[2-[2-[2-[[2-[2-[2-[[(4S)-4-carboxy-4-(1 7-carboxyheptadecanoylamino)butanoyl]amino]ethoxy]ethoxy]a cetyl]amino]ethoxy]ethoxy]acetyl]-lysine

SEQ ID NO: 20 - protein, 30 aa, synthetic construct; identical to SEQ ID NO: 1
  SITE 1: note = N-3-(imidazol-4-yl)-propionyl-tryptophan

SEQ ID NO: 21 - protein, 30 aa, synthetic construct; identical to SEQ ID NO: 2
  SITE 1: note = N-3-(imidazol-4-yl)-propionyl-glycine

SEQ ID NO: 22 - protein, 31 aa, synthetic construct; identical to SEQ ID NO: 5
  SITE 31: note = N{Epsilon}-[(2S)-2,6-bis[[2-[2-[2-[[2-[2-[2-[[2-[2- [2-[[2-[2-[2-[[2-[2-[2-[[2-[2-[2-[[(4S)-4-carboxy-4-[[4-[( 19-carboxynonadecanoylamino)methyl]cyclohexanecarbonyl]ami no]butanoyl]amino]ethoxy]ethoxy]acetyl]amino]ethoxy]ethoxy ]acetyl]amino]ethoxy]ethoxy]acetyl]amino]ethoxy]ethoxy]ace tyl]amino]ethoxy]ethoxy]acetyl]amino]ethoxy]ethoxy]acetyl] amino]hexanoyl]-lysine

(19) United States
(12) Patent Application Publication
(10) Pub. No.: US 2026/0125445 A1
(43) Pub. Date: May 7, 2026

(54) GLP-1 DERIVATIVES AND USES THEREOF

(71) Applicant: Novo Nordisk A/S, Bagsvaerd (DK)

(72) Inventors: Lars Linderoth, Hilleroed (DK); Steffen Reedtz-Runge, Birkeroed (DK); Maria Waldhoer, Hertford (GB); Jacob Kofoed, Vaerloese (DK)

(21) Appl. No.: 19/359,766

(22) Filed: Oct. 16, 2025

Related U.S. Application Data
(63) Continuation of application No. PCT/EP2025/069132, filed on Jul. 4, 2025.

(30) Foreign Application Priority Data
Jul. 5, 2024 (EP) 24186705.0

(51) Int. Cl. C07K 14/605; A61K 38/00; A61P 3/04

(52) U.S. Cl. CPC C07K 14/605; A61P 3/04; A61K 38/00

(57) ABSTRACT

The present invention relates to novel GLP-1 derivatives that are activating the GLP-1 receptor in a way that favours a cAMP response and has an impaired beta-arrestin recruitment. More particular the present invention relates to such GLP-1 derivatives and their use for treatment of diseases such as diabetes and obesity.

### TECHNICAL FIELD

The present invention relates to novel GLP-1 derivatives, and their pharmaceutical use. Furthermore, the invention relates to pharmaceutical compositions comprising such GLP-1 derivatives, and the use of such compounds for the treatment of medical conditions relating to diabetes and obesity.

### CROSS-REFERENCE TO RELATED APPLICATIONS

This application is a continuation of International Application PCT/EP2025/069132, filed Jul. 4, 2025, which claims priority to European Patent Application 24186705.0 filed Jul. 5, 2024, the contents of which are incorporated by reference in their entirety.

### INCORPORATION-BY-REFERENCE OF THE SEQUENCE LISTING

The instant application contains a Sequence Listing which has been submitted in XML format via USPTO patent electronic filing system and is hereby incorporated by reference in its entirety. Said XML file, created on Oct. 1, 2025, is named “240010US01.xml”, and is 36,490 bytes in size.

### BACKGROUND

Glucagon-like peptide 1 (GLP-1), analogues and derivatives thereof, are known for their ability to stimulate insulin secretion, induce weight loss due to reduction of appetite, slowing gastric emptying, improve cardiovascular, liver and kidney health. They have been used for the treatment of e.g. type 2 diabetes and obesity for some years with one of the most recent derivatives being semaglutide present in products Ozempic® and WeGovy® (WO2006/097537).

GLP-1 acts by interacting with the GLP-1 Receptor which is a G-protein coupled receptor (GPCR) expressed in several tissues and cell types including pancreatic beta cells (resulting in insulin secretion) and neurons in the brain (resulting in food intake suppression). When stimulated GPCRs affect several signalling pathway, e.g. G's-protein pathway resulting in cAMP production and beta-arrestin leading to receptor internalization and potentially receptor mediated clearance of the ligand. Some GLP-1 analogues and derivatives thereof may activate the signalling pathways demonstrating a difference in potency/efficacy between the pathways. This concept is referred to as biased GLP-1 agonism. Thus, some GLP-1 analogues can activate both cAMP and beta-arrestin pathways with a maximum efficacy while other GLP-1 analogues would fully activate cAMP pathway without the full activation of beta-arrestin pathway. Preclinical evidence indicates that biased GLP-1 analogues or derivatives with reduced beta-arrestin signalling can provide a better weight loss and glucose regulation with a similar or better tolerability profile compared to balanced GLP-1 analogues or derivatives like semaglutide. Based on genetic evidence, low frequency loss-of-function (LoF) variants in beta-arrestin (ARRB1) are associated with improved HbA1c regulation in patients with T2D treated with GLP-1 receptor agonists (Dawed et al, Lancet Diabetes Endocrinol, 2023 January; 11(1):33-41). Therefore, it has been suggested that biased GLP-1 compounds favouring cAMP generation over beta-arrestin recruitment may lead to a higher benefit for a broader patient population mimicking the improved GLP-1 response in patients with beta-arrestin LoF variants. While weight loss can partially explain the benefit of GLP-1 in obesity related comorbidities (e.g. cardiovascular disease), biased GLP-1 action through other cells with reduced beta-arrestin signalling expressed in all tissues in the body, can hypothetically provide additional benefits for patients with cardiometabolic diseases.

GLP-1 derivatives and biased GLP-1 derivatives are disclosed in e.g. WO2009/030738, WO2017/149070, WO2016/161244 and EP3783014. However, there is still a need for development of GLP-1 derivative and analogues having a biased signalling with high activation of the cAMP pathway and impaired beta-arrestin recruitment.

### SUMMARY

The invention relates to derivatives of GLP-1 analogues that have an imidazolepropionyl at a position corresponding to position 7 of hGLP-1(7-37) (SEQ ID NO: 11) and Trp or Gly at a position corresponding to position 8 of hGLP-1(7-37). The derivatives have a substituent attached to a Lys residue of the GLP-1 analogue at a position corresponding to position 37 of hGLP-1 (7-37). The invention also relates to corresponding GLP-1 analogues that have an imidazolepropionyl at a position corresponding to position 7 of hGLP-1(7-37) (SEQ ID NO: 11) and Trp or Gly at a position corresponding to position 8 of hGLP-1(7-37).

Furthermore, the invention relates to pharmaceutical compositions comprising such GLP-1 derivatives and pharmaceutically acceptable excipients, as well as the medical use of said derivatives.

In a first aspect, the invention relates to derivatives of GLP-1 analogues that are capable of activating the GLP-1 receptor by inducing a cAMP response, while showing a reduced beta-arrestin signalling. In a further aspect, the GLP-1 derivatives of the invention are selectively activating the GLP-1 receptor over the GIP receptor.

Also, or alternatively, in a second aspect, the invention relates to GLP-1 derivatives with improved pharmacokinetic properties.

Also, or alternatively, in a third aspect, the invention relates to GLP-1 derivatives that are physically stable.

Also, or alternatively, in a fourth aspect, the invention relates to GLP-1 derivatives that show improved chemical stability.

### BRIEF DESCRIPTION OF DRAWINGS

FIG. 1 shows concentration response curves for beta-arrestin2 recruitment to GLP-1R for Compound 1, hGLP-1(7-37) and semaglutide and that the efficacy (Eₘₐₓ) is significantly reduced for Compound 1 of the present invention.

### DESCRIPTION

In what follows, Greek letters may be represented by their symbol or the corresponding written name, for example: α=alpha; β=beta; ε=epsilon; γ=gamma; ω=omega; etc. Also, the Greek letter of may be represented by “u”, e.g. in μl=ul, or in μM=uM.

An asterisk (*) or a waved line in a chemical formula designates i) a point of attachment, ii) a radical, and/or iii) an unshared electron.

Any intervals disclosed herein is generally closed, i.e. the end points are included. For example, the number of consecutive —CH₂-groups in the substituent attached to a lysine residue of the invention is in the range of 14-18, which means from 14 to 18, both are inclusive.

Unless otherwise indicated in the specification, terms presented in singular form generally also include the plural situation.

Also described herein are derivatives, derivatives of GLP-1 analogues, methods of preparation, and pharmaceutical compositions and uses as disclosed herein, wherein open ended terms like “comprises” and “comprising” are replaced with closed terms such as “consists of”, “consisting of”, and the like.

The invention relates to derivatives of GLP-1 analogues that have an imidazolepropionyl at a position corresponding to position 7 of hGLP-1(7-37) (SEQ ID NO: 11) and Trp or Gly at a position corresponding to position 8 of hGLP-1(7-37). The derivatives have a substituent attached to a Lys residue of the GLP-1 analogue at a position corresponding to position 37 of hGLP-1 (7-37). The invention also relates to corresponding GLP-1 analogues that have an imidazolepropionyl at a position corresponding to position 7 of hGLP-1(7-37) (SEQ ID NO: 11) and Trp or Gly at a position corresponding to position 8 of hGLP-1(7-37).

Furthermore, the invention relates to pharmaceutical compositions comprising such GLP-1 derivatives and pharmaceutically acceptable excipients, as well as the medical use of said derivatives.

In a first aspect, the invention relates to derivatives of GLP-1 analogues that are capable of activating the GLP-1 receptor by inducing a cAMP response, while showing a reduced beta-arrestin signalling. In a further aspect, the GLP-1 derivatives of the invention are selectively activating the GLP-1 receptor over the GIP receptor.

Also, or alternatively, in a second aspect, the invention relates to GLP-1 derivatives with improved pharmacokinetic properties.

Also, or alternatively, in a third aspect, the invention relates to GLP-1 derivatives that are physically stable.

Also, or alternatively, in a fourth aspect, the invention relates to GLP-1 derivatives that show improved chemical stability.

### Compounds

### GLP-1 Receptor Agonist

A receptor agonist may be defined as a compound that binds to a receptor and elicits a response typical of the natural ligand (see e.g. “Principles of Biochemistry”, A L Lehninger, D L Nelson, M M Cox, Second Edition, Worth Publishers, 1993, page 763).

As described herein, a “GLP-1 receptor agonist” may be defined as a compound which is capable of binding to the GLP-1 receptor and capable of activating it.

### GLP-1 Analogues

The term “hGLP-1(7-37)” as used herein refers to the human glucagon-like peptide-1, the sequence of which is included in the sequence listing as SEQ ID NO: 11. The peptide having SEQ ID NO: 11 may also be designated “native GLP-1” or simply “GLP-1”.

The term “GLP-1 analogue” as used herein refers to a peptide, or a compound which is a variant of hGLP-1(7-37). The term “variant” is used for peptides comprising at least one amino acid substitution as compared to hGLP-1(7-37) and which is capable of activating the GLP-1 receptor.

The GLP-1 analogue of the invention has an imidazolepropionyl at a position corresponding to position 7 of GLP-1(7-37), such as a 3-(imidazol-4-yl)-propionyl or 3-imidazol-2-yl. In one embodiment the imidazolepropionyl is 3-(imidazol-4-yl)propionyl, also known as deamino-histidine or abbreviated as “Imp”.

The numbering of amino acids residues (such as “position 7”) in the GLP-1 analogues of the invention follows established practice in the art for GLP-1, namely that the first amino acid residue is numbered or accorded position no. 7, and the subsequent amino acid residues downstream towards the C-terminus are numbered 8, 9, 10 and so on, until the last (C-terminal) amino acid residue. Imidazolepropionyl (Imp) will be described as being part of the peptide backbone, even though it is not an amino acid.

The numbering is done differently in the sequence listing, where the first amino acid residue of SEQ ID NO: 11 (His) is assigned no. 1. However, herein we follow the established numbering practice in the art. In the sequence listing, sequences with Imp at a position corresponding to position 7 of GLP-1(7-37) (e.g. SEQ ID NO: 1), Imp is not part of the sequence listing as it is not an amino acid, but is described as a N-acylation of the amino acid in a position corresponding to position 8 of GLP-1(7-37), which thus become amino acid no. 1 in the sequence listing.

In one aspect, the GLP-1 analogue of the invention is a peptide of Formula I (SEQ ID NO: 18):

Imp-X₈EGTFTSDVSSYLEEQAAREFIAWLVRGRK,

wherein Imp is 3-(imidazol-4-yl)-propionyl, and
wherein X₈ is W or G; or a pharmaceutically acceptable salt, amide or ester thereof.

In some embodiment, the GLP-1 analogue of the invention is a peptide of SEQ ID NO: 20; or a pharmaceutically acceptable salt, amide or ester thereof. Also or alternatively, in some embodiment, the GLP-1 analogue of the invention is a peptide of SEQ ID NO: 21; or a pharmaceutically acceptable salt, amide or ester thereof.

The GLP-1 analogues of the invention may be described by reference to i) the number of the amino acid residue in hGLP-1(7-37) which corresponds to the amino acid residue which is changed (i.e., the corresponding position in hGLP-1(7-37)), and to ii) the actual change. For example, [Imp7, Trp8, Glu22, Arg26, Arg34, Lys37]-hGLP-1(7-37) refers to a GLP-1 analogue in which positions corresponding to positions 7, 8, 22, 26, 34, and 37 of hGLP-1(7-37) each have been substituted for the mentioned amino acid, i.e. a peptide of SEQ ID NO: 9.

The term “substitution” as used herein refers to one amino acid being replaced by another amino acid or a chemical building block that can be incorporated into the backbone of a peptide. In one aspect, amino acids may be substituted by conservative substitution. The term “conservative substitution” as used herein denotes that one or more amino acids are replaced by another, biologically similar residue. Examples include substitution of amino acid residues with similar characteristics, e.g. small amino acids, acidic amino acids, polar amino acids, basic amino acids, hydrophobic amino acids and aromatic amino acids. In one aspect, the GLP-1 analogues of the derivatives of the invention may comprise substitutions of one or more unnatural and/or non-amino acids, e.g., amino acid mimetics, into the sequence of the GLP-1 analogue.

Analogues “comprising” certain specified changes may comprise further changes, when compared to the respective formula. In a particular embodiment, the analogue “has” the specified changes.

As is apparent from the above examples, amino acid residues may be identified by their full name, their one-letter code, and/or their three-letter code. These three ways are fully equivalent.

In what follows, all amino acids of the GLP-1 analogues of the invention for which the optical isomer is not stated is to be understood to mean the L-isomer (unless otherwise specified).

### GLP-1 Derivatives

The term “derivative” as used herein in the context of a GLP-1 analogue means a chemically modified GLP-1 analogue, in which one or more substituents have been covalently attached to the peptide backbone. In some embodiment, the substituent may be referred to as a side chain.

In one aspect, the GLP-1 analogue comprises a substituent covalently attached to the amino acid residue corresponding to position 37 of hGLP-1(7-37). In some embodiments, the substituent is capable of forming non-covalent conjugates with proteins, e.g. albumin, thereby promoting the circulation of the derivative with the blood stream, and also having the effect of protracting the time of action of the derivative, due to the fact that the conjugate of the GLP-1 derivative and albumin is only slowly disintegrated to release the active pharmaceutical ingredient.

The substituent may be covalently attached to a lysine residue of the GLP-1 analogue by acylation, i.e. via an amide bond formed between a carboxylic acid group of the modifying group and the epsilon amino group of said lysine group. The amino group of lysine could also be coupled to an aldehyde of the modifying group by reductive amination.

In one embodiment, the substituent is covalently attached to a lysine residue at a position corresponding to position 37 of hGLP1(7-37) by acylation, i.e. via an amide bond formed between a carboxylic acid group of the modifying group and the epsilon amino group of the lysine residue.

In one embodiment, the substituent is defined by Formula III:

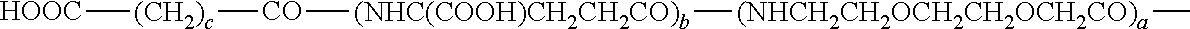

wherein a is 1-3, b is 1-2, and c is 14-18.

In some embodiment, the substituent is defined by Formula III wherein a is 2, b is 1 and c is 16, i.e. Formula Illa:

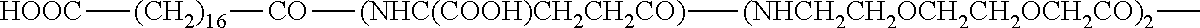

In some embodiment, the substituent is Chem. 1:

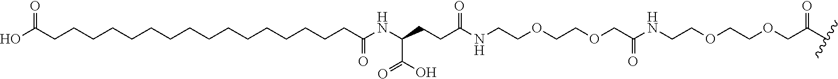 [SMILES: *C(=O)COCCOCCNC(=O)COCCOCCNC(=O)CC[C@H](NC(=O)CCCCCCCCCCCCCCCCC(=O)O)C(=O)O]

The derivatives of the invention may exist in different stereoisomeric forms having the same molecular formula and sequence of bonded atoms, but differing only in the three-dimensional orientation of their atoms in space. The stereoisomerism of the exemplified derivatives of the invention is indicated in the experimental section, in the names as well as the structures, using standard nomenclature. Unless otherwise stated the invention relates to all stereoisomeric forms of the claimed derivative.

The GLP-1 derivatives and analogues of the invention have GLP-1 activity. This term refers to the ability to bind to the GLP-1 receptor and initiate a signal transduction pathway resulting in insulinotropic action or other physiological effects as is known in the art. For example, the analogues and derivatives of the invention can be tested for GLP-1 activity using the assays as described in Examples 1-7.

### Pharmaceutically Acceptable Salt, Amide, or Ester

The derivatives of the invention may be in the form of a pharmaceutically acceptable salt, amide, or ester.

Salts are e.g. formed by a chemical reaction between a base and an acid, e.g.:

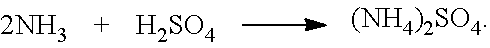

The salt may be a basic salt, an acid salt, or it may be neither nor (i.e. a neutral salt). Basic salts produce hydroxide ions and acid salts hydronium ions in water.

The salts of the derivatives of the invention may be formed with added cations or anions between anionic or cationic groups, respectively. These groups may be situated in the peptide moiety, and/or in the modifying group of the derivatives of the invention.

Non-limiting examples of anionic groups of the derivatives of the invention include free carboxylic groups in the side chain, if any, as well as in the peptide moiety. The peptide moiety often includes a free carboxylic acid group at the C-terminus, and it may also include free carboxylic groups at internal acid amino acid residues such as Asp and Glu.

Non-limiting examples of cationic groups in the peptide moiety include the free amino group at the N-terminus, if present, as well as any free amino group of internal basic amino acid residues such as His, Arg, and Lys.

The amide of the derivatives of the invention may, e.g., be formed by the reaction of a free carboxylic acid group with an amine or a substituted amine, or by reaction of a free or substituted amino group with a carboxylic acid.

The amide formation may involve the free carboxylic group at the C-terminus of the peptide, any free carboxylic group in the side chain, the free amino group at the N-terminus of the peptide, and/or any free or substituted amino group of the peptide in the peptide and/or the side chain.

The ester of the derivative or analogue of the invention, may e.g. be formed by the reaction of a free carboxylic acid group with an alcohol or a phenol, which leads to replacement of at least one hydroxyl group by an alkoxy or aryloxy group.

The ester formation may involve the free carboxylic acid group of the C-terminus of the peptide, and/or any free carboxylic group in the amino acid side chain.

In one aspect, the derivative of the invention is in the form of a pharmaceutically acceptable salt. Also, or alternatively, in one aspect, the derivative of the invention is in the form of a pharmaceutically acceptable amide, preferably with an amide group at the C-terminus of the peptide. Also or alternatively, in some embodiments, the derivative of the invention is in the form of a pharmaceutically acceptable ester.

### Functional Properties

In a first functional aspect, the derivatives of the invention have a good potency at the GLP-1 receptor. Also, or alternatively, in a second functional aspect, the derivatives of the invention selectively activate the GLP-1 receptor over the GIP receptor. Also, or alternatively, in a third functional aspect, the derivatives of the invention have low efficacy towards beta-arrestin recruitment. Also, or alternatively, in a fourth functional aspect, the derivatives of the invention have good pharmacokinetic properties. Also, or alternatively, in a fifth functional aspect, the derivatives of the invention are physically stable. Also, or alternatively, in a sixth functional aspect, the derivatives of the invention are chemically stable.

### Biological Activity—In Vitro Activity

According to the first functional aspect, the derivatives of the invention, as well as the constituent GLP-1 analogues, are biologically active, or potent at the human GLP-1 receptor.

In one embodiment, potency and/or activity refers to in vitro potency, i.e. performance in a functional GLP-1 receptor assay, more in particular to the capability of activating the human GLP-1 receptor. In one embodiment, the derivative of the invention is capable of inducing a cAMP generation upon activation of the GLP-1 receptor.

The in vitro potency may, e.g., be determined in a medium containing membranes expressing the human GLP-1 receptor, and/or in an assay with whole cells expressing the human GLP-1 receptor.

For example, the response of the human GLP-1 receptor may be measured in a reporter gene assay, e.g. in a stably transfected BHK cell line that expresses the human GLP-1 receptor and contains the DNA for the cAMP response element (CRE) coupled to a promoter and the gene for firefly luciferase (CRE luciferase). When cAMP is produced as a result of activation of the GLP-1 receptor this in turn results in the luciferase being expressed. Luciferase may be determined by adding luciferin, which by the enzyme is converted to oxyluciferin and produces bioluminescence, which is measured and is a measure of the in vitro potency. One non-limiting example of such an assay is described in Example 2 as described herein.

The term half maximal effective concentration (EC₅₀) generally refers to the concentration which induces a response halfway between the baseline and maximum, by reference to the dose response curve. EC₅₀ is used as a measure of the potency of a compound and represents the concentration where 50% of its maximal effect is observed.

The in vitro potency of the derivatives of the invention may be determined as described above, and the EC₅₀ of the derivative in question determined. The lower the EC₅₀ value, the better the potency. The EC₅₀ may be determined as an absolute value or relative to a reference compound.

In a further particular embodiment, the derivative of the invention has an absolute in vitro potency determined using the method of Example 2 in the absence of HSA corresponding to an EC₅₀ at or below 50 pM, more preferably below 40 pM, even more preferably below 30 pM, or most preferably below 25 pM. Also, or alternatively, in a further particular embodiment, the derivative of the invention has an absolute in vitro potency determined using the method of Example 2 in the presence of 1% HSA corresponding to an EC₅₀ at or below 1000 pM, more preferably below 800 pM, or even more preferably below 700 pM.

According to the second functional aspect, the derivatives of the invention are capable of activating the human GLP-1 receptor selectively over the human GIP receptor. The term “selectively” when used in relation to activation of the GLP-1 receptor over the GIP receptor refers to derivatives that display at least 10 fold, such as at least 50 fold, at least 500 fold, or at least 1000 fold better absolute potency for the GLP-1 receptor over the GIP receptor as measured in vitro in a potency assay for receptor function, such as an CRE luciferase functional potency assay without HSA, and compared by EC₅₀ values. The term “better potency” of the derivatives of the invention at the GLP-1 receptor over the GIP receptor is determined by the ratio of the EC₅₀ values at the GIP receptor versus GLP-1 receptor, respectively.

In a third functional aspect, the derivatives of the invention show reduced beta-arrestin recruitment at the GLP-1 receptor compared to a reference GLP-1 agonist, such as GLP-1 or semaglutide. In one embodiment, the derivative of the invention is activating the GLP-1 receptor by activating the cAMP, while showing reduced beta-arrestin recruitment. This has also referred to as being bias at the GLP-1 receptor.

In some embodiment, the derivative of the invention has a low Eₘₐₓ (effect maximum) in percent compared to a reference compound, such as hGLP-1(7-37). For example, the Eₘₐₓ may be determined in vitro in whole cells expressing the human GLP-1 receptor and expressing a split Luciferase with the Large BiT component fused to the GLP-1R C-terminus and the Small BIT fused to one of the 2 isoforms of beta-arrestin, beta-arrestin1 or beta-arrestin2. When the GLP-1R is activated, it causes translocation of beta-arrestin to the receptor, the subsequent association of Large BiT and Small BiT forms a functional luciferase whose activity can be detected by addition of a substrate. One non-limiting example of such an assay is described in Example 4 as described herein.

In one embodiment, the derivative of the invention has significantly reduced efficacy in a beta-arrestin assay, for both isoform 1 & 2, such as in the assay described in Example 4 herein. In a particular embodiment, the derivative of the invention has an Eₘₐₓ of less than 50% relative to hGLP-1(7-37) or more preferably less than 40% at both the beta-arrestin1 and beta-arrestin2.

### Pharmacokinetic Properties—Half-Life In Vivo in Minipigs

According to the fourth functional aspect, the derivatives of the invention have improved pharmacokinetic properties such as increased terminal half-life.

Increasing terminal half-life means that the compound in question is eliminated slower from the body. For the derivatives of the invention this entails an extended duration of the pharmacological effect.

The pharmacokinetic properties of the derivatives of the invention may suitably be determined in vivo in pharmacokinetic (PK) studies. Such studies are conducted to evaluate how pharmaceutical compounds are absorbed, distributed, and eliminated in the body, and how these processes affect the concentration of the compound in the body, over the course of time.

In the discovery and preclinical phase of pharmaceutical drug development, animal models such as the mouse, rat, monkey, dog, or pig, may be used to perform this characterisation. Any of these models can be used to test the pharmacokinetic properties of the derivatives of the invention.

In such studies, animals are typically administered with a single dose of the drug, either intravenously (i.v.), subcutaneously (s.c.), or orally (p.o.) in a relevant formulation. Blood samples are drawn at predefined time points after dosing, and samples are analysed for concentration of drug with a relevant quantitative assay. Based on these measurements, time-plasma concentration profiles for the compound of study are plotted and a so-called non-compartmental pharmacokinetic analysis of the data is performed.

For most compounds, the terminal part of the plasma-concentration profiles will be linear when drawn in a semi-logarithmic plot, reflecting that after the initial absorption and distribution, drug is removed from the body at a constant fractional rate. The rate (lambda Z or λ_{z}) is equal to minus the slope of the terminal part of the plot. From this rate, also a terminal half-life may be calculated, as t½=ln(2)/λ_{z} (see, e.g., Johan Gabrielsson and Daniel Weiner: Pharmacokinetics and Pharmacodynamic Data Analysis. Concepts & Applications, 3rd Ed., Swedish Pharmaceutical Press, Stockholm (2000)).

Clearance can be determined after i.v. administration and is defined as the dose (D) divided by area under the curve (AUC) on the plasma concentration versus time profile (Rowland, M and Tozer T N: Clinical Pharmacokinetics: Concepts and Applications, 3^{rd} edition, 1995 Williams Wilkins).

The estimate of terminal half-life and/or clearance is relevant for evaluation of dosing regimens and an important parameter in drug development, in the evaluation of new drug compounds.

According to the fourth functional aspect, the derivatives of the invention have improved pharmacokinetic properties.

In a particular embodiment, the pharmacokinetic properties may be determined as terminal half-life (t_{1/2}) in vivo in minipigs after i.v. administration, e.g. as described in Example 5 herein.

In particular embodiments, the terminal half-life in minipigs is at least 75 hours, preferably at least 100 hours, even more preferably at least 140 hours. The long half-life in minipigs suggests that the derivatives of the invention may be suitable for administration in humans with a frequency of once-weekly or lower, such as in twice-monthly, or once-monthly dosing regimen. In one embodiment, the derivatives of the invention have a long half-life in humans suitable for once-weekly dosing.

### Physical Properties

According to the fifth functional aspect, the derivative of the invention has improved physical stability in solution. The term “physical stability” refers to the tendency of the polypeptide to form biologically inactive and/or insoluble aggregates, e.g. amyloid fibrils or gels.

In a particular embodiment, the improved physical stability may be determined by measuring lag-time and/or recovery in a Thioflavin T (ThT) fibrillation assay, e.g. as described in Example 6 herein.

In a further particular embodiment, the derivative of the invention has a lag-time in the ThT fibrillation assay of more than 20 hours, preferably more than 30 hours, even more preferably 45 hours or more, such as shown in Example 6 described herein.

In a further particular embodiment, the derivative of the invention has more than 80 percent recovery in a ThT fibrillation assay, such as shown in Example 6 described herein. A recovery above 80 percent is considered full recovery.

### Chemical Properties

According to the sixth functional aspect, the derivatives of the invention have improved chemical stability. The term “chemical stability” refers to chemical (in particular covalent) changes in the polypeptide structure leading to formation of chemical degradation products, such as high molecular weight proteins (HMWPs), deamidation, isomerization and hydrolysis products potentially having a reduced biological potency, and/or increased immunogenic effect as compared to the intact polypeptide.

In a particular embodiment, the improved chemical stability may be determined by measuring the content of HMWP and/or purity loss, by measuring the amount of chemical degradation products at various time-points after exposure to different environmental conditions, e.g. by SEC-HPLC, and/or LCMS, e.g. as described in Example 7 herein.

In a further particular embodiment, the derivative of the invention has a purity loss over 4 weeks of less than 10 percent, preferably less than 7 percent, more preferably less than 4 percent, such as shown in Example 7 described herein.

In a further particular embodiment, the derivative of the invention has a formation of HMWP's over 4 weeks of less than 4 percent, preferably less than 2 percent, more preferably less than 1 percent, such as shown in Example 7 described herein.

Additional particular embodiments of the derivatives of the invention are described in the section headed “particular embodiments”.

### Production Processes

The production of peptides like hGLP-1 and hGLP-1 analogues is well known in the art.

The GLP analogues of the derivatives of the invention (or fragments thereof) as well as the GLP-1 analogues of the invention, may for instance be produced by classical peptide synthesis, e.g., solid phase peptide synthesis using t-Boc or Fmoc chemistry or other well established techniques, see, e.g., Greene and Wuts, “Protective Groups in Organic Synthesis”, John Wiley & Sons, 1999, Florencio Zaragoza Dörwald, “Organic Synthesis on solid Phase”, Wiley-VCH Verlag GmbH, 2000, and “Fmoc Solid Phase Peptide Synthesis”, Edited by W. C. Chan and P. D. White, Oxford University Press, 2000.

Also, or alternatively, in some embodiments, the entire GLP-1 analogue of the invention, or the entire GLP-1 analogue part of the derivative of the invention, is produced by recombinant methods, viz. by culturing a host cell containing a DNA sequence encoding the analogue and capable of expressing the peptide in a suitable nutrient medium under conditions permitting the expression of the peptide. Non-limiting examples of host cells suitable for expression of these peptides are: Escherichia coli, Saccharomyces cerevisiae, as well as mammalian BHK or CHO cell lines.

Those derivatives of the invention which include non-natural amino acids and/or a covalently attached N-terminal mono- or dipeptide mimetic may e.g. be produced as described in the experimental part. Or see e.g., Hodgson et al: “The synthesis of peptides and proteins containing non-natural amino acids”, Chemical Society Reviews, vol. 33, no. 7 (2004), p. 422-430.

Specific examples of methods of preparing the derivatives of the invention are included in the experimental part.

### Pharmaceutical Compositions

The invention also relates to pharmaceutical compositions comprising a derivative of the invention or a pharmaceutically acceptable salt, amide, or ester thereof, and a pharmaceutically acceptable excipient. Such compositions may be prepared as is known in the art.

The term “excipient” broadly refers to any component other than the active therapeutic ingredient(s). The excipient may be an inert substance, an inactive substance, and/or a not medicinally active substance.

The excipient may serve various purposes, e.g. as a carrier, vehicle, diluent, tablet aid and/or to improve administration, and/or absorption of the active substance.

The formulation of pharmaceutically active ingredients with various excipients is known in the art, see e.g. Remington: The Science and Practice of Pharmacy (e.g. 19^{th} edition (1995), and any later editions).

Non-limiting examples of excipients are: Solvents, diluents, buffers, preservatives, tonicity regulating agents, chelating agents, and stabilizers.

Examples of formulations include liquid formulations, i.e. aqueous formulations comprising water. A liquid formulation may be a solution, or a suspension. An aqueous formulation typically comprises at least 50% w/w water, or at least 60%, 70%, 80%, or 90% w/w of water.

### Pharmaceutical Indications

The present invention also relates to a derivative or analogue of the invention for use as a medicament.

In some embodiments, the derivative or analogue of the invention may be used for the following medical treatments:
(i) Prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1c;
(ii) Delaying or preventing diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes;
(iii) the prevention and/or treatment of certain eating disorders, overweight and/or obesity; e.g. by decreasing food intake, suppressing appetite, inducing satiety, reducing body weight; treating or preventing binge eating disorder, food cravings, bulimia nervosa and/or obesity induced by medication, such as an antipsychotic or a steroid; reducing gastric motility; and/or delaying gastric emptying;
(iv) the prevention and/or treatment of cardiovascular disease, such as the delaying or reduction of the development of a major adverse cardiovascular event (MACE) selected from the group consisting of cardiovascular death, non-fatal myocardial infarction, non-fatal stroke, revascularisation, hospitalisation for unstable angina pectoris, and hospitalisation for heart failure;
(v) the prevention and/or treatment of non-alcoholic fatty liver disease (NAFLD) and/or non-alcoholic steatohepatitis (NASH), otherwise known as metabolic dysfunction-associated steatohepatitis (MASH), and/or alcoholic liver disease (ALD);
(vi) the prevention and/or treatment of chronic kidney disease;
(vii) the prevention and/or treatment of cognitive disorders including dementia and Alzheimer's disease.

The indication may be (i). The indication may be (ii). The indication may be (iii). The indication may be (iv). The indication may be (v). The indication may be (vi). The indication may be (vii). The indication may be overweight or obesity. The indication may be type 2 diabetes.

Generally, all subjects suffering from obesity are also considered to be suffering from overweight. The subject suffering from obesity may be a human being, such as an adult human or a child, wherein “child” includes the infant and the adolescents.

The World Health Organisation (WHO) defines obesity as being the abnormal or excessive accumulation of fat that may impair health and considers body mass index (BMI) to be the most convenient population-level measure of overweight and obesity. The formula used to calculate BMI is weight in kilograms (kg)/height in meters squared (m²).

For adults, the WHO defines overweight and obesity as follows: overweight means having a BMI greater than or equal to 25; obesity means having a BMI greater than or equal to 30.

For children, the WHO considers age when defining overweight and obesity.

For children under the age of five, overweight means having a weight-for-height greater than two standard deviations above the WHO Child Growth Standards median; and obesity means having a weight-for-height greater than three standard deviations above the WHO Child Growth Standards median.

Overweight and obesity are defined as follows for children aged five to nineteen: overweight means having a BMI-for-age that is greater than one standard deviation above the WHO Growth Reference median; and obesity means having a BMI-for-age that is greater than two standard deviations above the WHO Growth Reference median.

Nonetheless, the diagnostic criteria for underweight, the normal range, pre-obesity/overweight and obesity can differ between countries/populations, as illustrated in Table (i) below for adults.

TABLE i
Definitions of underweight, the normal range,
pre-obesity/overweight and obesity in adults

| | BMI (kg/m²) | | | |
|---|---|---|---|---|
| | International/ | | | |
| Nutritional | European/US | Chinese | Japanese | Taiwanese |
| status | classification | population | classification | classification |
| Underweight | <18.5 | <18.5 | <18.5 | <18.5 |
| Normal weight | 18.5-24.9 | >/=18.5 and <23 | >/=18.5 and <25 | >/=18.5 and <24 |
| Pre-obesity/ | 25.0-29.9 | 24 ≤ BMI < 28 | | >/=24 and <27 |
| Overweight | | | | |
| Obesity | >/=30 | ≥28 | >/=25 | |
| Obesity class I | 30.0-34.9 | | >/=25 and <30 | 27 ≤ and < 30 |
| Obesity class II | 35.0-39.9 | | >/=30 and <35 | 30 ≤ and < 35 |
| Obesity class III | >/=40 | | >/=35 and <40 | ≥35 |
| Obesity class IV | | | >/=40 | |

Guidelines for the Asian population were published by Misra A et al. J Assoc Physicians India. 2009; 57:163-70.

Guidelines for the Chinese population were issued in the 2006 edition of the Guidelines for Prevention and Control of Overweight and Obesity in Chinese Adults, compiled by the Chinese Working Group on Obesity.

Guidelines for the Japanese population were issued, in 2016, by the Japanese Society for the Study of Obesity (JASSO) in Guidelines for the management of obesity disease.

Guidelines for the Taiwanese population were issued by the Taiwanese government's Health Promotion Administration (HPA), Ministry of Health and Welfare in 2023, in the 2^{nd} edition of its “Evidence-Based Guideline on Adult Obesity Prevention and Management”.

An adult human subject suffering from obesity may thus have a BMI of 25 kg/m² or more, 27 kg/m² or more, 28 kg/m² or more or 30 kg/m² or more; this subject may also be referred to as being obese. The obesity may be class I, class II, class Ill or class IV obesity. An adult human subject suffering from overweight may have a BMI of 24 kg/m² or more, 25 kg/m² or more, or 27 kg/m² or more. In some embodiments a human subject suffering from overweight has a BMI in the range of 24 to <27 kg/m², in the range of 24 to <28 kg/m², in the range of 25 to <30 kg/m² or in the range of 27 to <30 kg/m².

A higher than normal BMI increases the risk of an individual developing any one of a wide range of other diseases or co-morbidities. The weight-related co-morbidity may be one, or a combination of any one of the diseases mentioned in (i), (ii), (iv), and (v), above.

The GLP-1 derivative disclosed herein may be for use in the treatment or prevention of overweight, wherein the patient may have at least one weight-related co-morbidity. The GLP-1 derivative disclosed herein may be for use in the treatment or prevention of obesity, wherein the patient may have at least one weight-related co-morbidity.

The GLP-1 derivative disclosed herein may be used as an adjunct to a reduced-calorie diet and increased physical activity for chronic weight management, in a subject that is obese at the start of treatment.

The GLP-1 derivative disclosed herein may be used as an adjunct to a reduced-calorie diet and increased physical activity for chronic weight management in a subject that is overweight at the start of treatment and has at least one weight-related co-morbidity.

The GLP-1 derivative disclosed herein may be used as an adjunct to a reduced-calorie diet and increased physical activity for chronic weight management in an adult human being with an initial body mass index (BMI) of 30 kg/m² or more, 28 kg/m² or more, 27 kg/m² or more or 25 kg/m² or more.

The GLP-1 derivative disclosed herein may be used as an adjunct to a reduced-calorie diet and increased physical activity for chronic weight management in an adult human being that has an initial body mass index (BMI) of 25 kg/m² or more or 24 kg/m² or more and at least one weight-related co-morbidity.

Administration of the GLP-1 derivative disclosed herein may result in >15% weight loss, such as >20% weight loss, such as >25% weight loss, such as >30% weight loss, such as about 15-40% weight loss, such as about 20-35% weight loss, such as about 25-30% weight loss, within 26 weeks of the start of treatment.

Administration of the GLP-1 derivative disclosed herein disclosed herein may result in >15% weight loss, such as >20% weight loss, such as >25% weight loss, such as >30% weight loss, such as about 15-40% weight loss, such as about 20-35% weight loss, such as about 25-30% weight loss, within 26 weeks of the start of treatment.

The GLP-1 derivative disclosed herein may be for use in the treatment or prevention of diabetes, as described in (i) or (ii), above. The GLP-1 derivative disclosed herein may be for use in the treatment or prevention of diabetes and at least one diabetes-related co-morbidity, such as (iv) or (vi), above.

### NON-LIMITING EMBODIMENTS

The invention is further described by the following non-limiting embodiments:
1. A GLP-1 derivative comprising a GLP-1 analogue of Formula I (SEQ ID NO: 18):

Imp-X₈EGTFTSDVSSYLEEQAAREFIAWLVRGRK,

wherein Imp is 3-(imidazol-4-yl)-propionyl, and
wherein X₈ is W or G, and
wherein K at a position corresponding to position 37 of GLP-1(7-37) is chemically modified at the epsilon amino group with a substituent of formula III: HOOC—(CH₂)_{c}—CO—(NHC(COOH)CH₂CH₂CO)_{b}—(NHCH₂CH₂OCH₂CH₂OCH₂CO)ₐ—,
wherein a is 1-3, b is 1-2, and c is 14-18, and
wherein the C-terminal amino acid is optionally a C-terminal amide,
or a pharmaceutically acceptable salt thereof.
2. A GLP-1 derivative consisting of a GLP-1 analogue of Formula I (SEQ ID NO: 18):

Imp-X₈EGTFTSDVSSYLEEQAAREFIAWLVRGRK,

wherein Imp is 3-(imidazol-4-yl)-propionyl, and
wherein X₈ is W or G, and
wherein K at a position corresponding to position 37 of GLP-1(7-37) is chemically modified at the epsilon amino group with a substituent of formula III: HOOC—(CH₂)_{c}—CO—(NHC(COOH)CH₂CH₂CO)_{b}—(NHCH₂CH₂OCH₂CH₂OCH₂CO)ₐ—,
wherein a is 1-3, b is 1-2, and c is 14-18, and
wherein the C-terminal amino acid is optionally a C-terminal amide,
or a pharmaceutically acceptable salt thereof.
3. The GLP-1 derivative according to any one of embodiments 1 or 2, wherein X₈ is W.
4. The GLP-1 derivative according to embodiments 1 or 2, wherein X₈ is G.
5. The GLP-1 derivative according to any one of the preceding embodiments, wherein a is 2.
6. The GLP-1 derivative according to any one of embodiments 1-4, wherein a is 1.
7. The GLP-1 derivative according to any one of embodiments 1-4, wherein a is 3.
8. The GLP-1 derivative according to any one of the preceding embodiments, wherein b is 1.
9. The GLP-1 derivative according to any one of embodiments 1-7, wherein b is 2.
10. The GLP-1 derivative according to any one of the preceding embodiments, wherein c is 16.
11. The GLP-1 derivative according to any one of embodiments 1-9, wherein c is 14.
12. The GLP-1 derivative according to any one of embodiments 1-9, wherein c is 18.
13. The GLP-1 derivative according to any one of the preceding embodiments, wherein the substituent is of formula IIIa: HOOC—(CH₂)₁₆—CO—(NHC(COOH)CH₂CH₂CO)—(NHCH₂CH₂OCH₂CH₂OCH₂CO)₂—.
14. The GLP-1 derivative according to any one of the preceding embodiments, wherein the substituent is Chem.1:

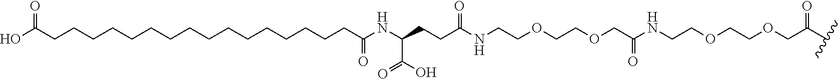 [SMILES: *C(=O)COCCOCCNC(=O)COCCOCCNC(=O)CC[C@H](NC(=O)CCCCCCCCCCCCCCCCC(=O)O)C(=O)O]

15. The GLP-1 derivative according to any one of the preceding embodiments, wherein the derivative is Compound 1:

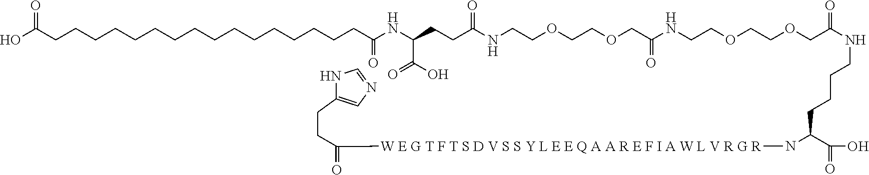

16. The GLP-1 derivative according to any one of the preceding embodiments, wherein the derivative is Compound 2:

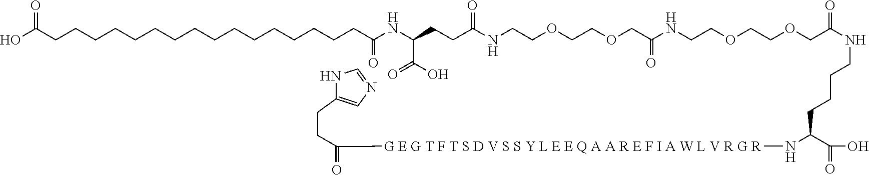

17. The GLP-1 derivative according to any one of the preceding embodiments, wherein the derivative is an agonist at the human GLP-1 receptor.
18. The derivative according to any one of the preceding embodiments, wherein the derivative is capable of activating the human GLP-1 receptor and result in cAMP production.
19. The derivative according to any one of the preceding embodiments, wherein the derivative is capable of activating the human GLP-1 receptor in an assay with whole cells expressing the human GLP-1 receptor.
20. The derivative according to any one of the preceding embodiments, wherein the derivative is capable of activating the human GLP-1 receptor in a CRE luciferase assay, such as Example 2 described herein.
21. The GLP-1 derivative according to any one of the preceding embodiments, wherein the derivative is selective for the human GLP-1 receptor over the human GIP receptor.
22. The GLP-1 derivative according to any one of the preceding embodiments, wherein the derivative has a lower EC₅₀ at the human GLP-1 receptor than at the human GIP receptor.
23. The GLP-1 derivative according to any one of the preceding embodiments, wherein the derivative has a lower EC₅₀ at the human GLP-1 receptor than at the human GIP receptor in a cAMP generating assay.
24. The GLP-1 derivative according to any one of the preceding embodiments, wherein the derivative has an EC₅₀ at the human GIP receptor of more than 10,000 pM in a CRE luciferase assay, such as determined in Example 3 described herein.
25. The GLP-1 derivative according to any one of the preceding embodiments, wherein the derivative has impaired beta-arrestin recruitment relative to hGLP-1(7-37)
26. The GLP-1 derivative according to any one of the preceding embodiments, wherein the derivative has a beta-arrestin Eₘₐₓ of <50% relative to hGLP-1 (7-37), such as determined in Example 4 describe herein.
27. The GLP-1 derivative according to any one of the preceding embodiments, wherein the derivative has a beta-arrestin Eₘₐₓ of <45% relative to hGLP-1 (7-37), such as determined in Example 4 describe herein.
28. The GLP-1 derivative according to any one of the preceding embodiments, wherein the derivative has a beta-arrestin Eₘₐₓ of <40% relative to hGLP-1 (7-37), such as determined in Example 4 describe herein.
29. The GLP-1 derivative according to any one of the preceding embodiments, wherein the derivative has improved pharmacokinetic properties.
30. The GLP-1 derivative according to anyone of the preceding embodiments, wherein the derivative has an increased half-life.
31. The GLP-1 derivative according to any one of the preceding embodiments, wherein the derivative has an increased half-life when determined in minipigs, such as determined in Example 5 described herein.
32. The GLP-1 derivative according to any one of the preceding embodiments, wherein the GLP-1 derivative has a half-life in humans suitable for once-weekly administration.
33. The GLP-1 derivative according to any one of the preceding embodiments, wherein the derivative has improved physical stability.
34. The GLP-1 derivative according to any one of the preceding embodiments, wherein the derivative has more than 80% recovery in the ThT fibrillation assay, such as determined in Example 6 described herein.
35. The GLP-1 derivative according to any one of the preceding embodiments, wherein the derivative has 45 hour or more in a ThT fibrillation assay, such as determined in Example 6 herein.
36. The GLP-1 derivative according to any one of the preceding embodiments, wherein the derivative has improved chemical stability.
37. The GLP-1 derivative according to any one of the preceding embodiments, wherein the derivative has a formation of HMWP of less than 1% over 4 weeks, such as determined in Example 7 described herein.
38. The GLP-1 derivative according to any one of the preceding embodiments, wherein the derivative has a purity loss of less than 1.5% over 4 weeks, such as determined in Example 7 described herein.
39. A GLP-1 analogue of SEQ ID NO: 18.
40. The GLP-1 analogue of embodiment 39, wherein the analogue is of SEQ ID NO: 20 or SEQ ID NO: 21.
41. A pharmaceutical composition comprising the GLP-1 derivative or analogue according to any one of the preceding embodiments and at least one pharmaceutically acceptable excipients.
42. A GLP-1 derivative or analogue according to any one of embodiments 1-40 or a composition according to embodiment 41, for use as a medicament.
43. A GLP-1 derivative or analogue according to any one of embodiments 1-40 or a composition according to embodiment 41, for use in the prevention or treatment of:
   (i) Prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1c;
   (ii) Delaying or preventing diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes;
   (iii) the prevention and/or treatment of certain eating disorders, overweight and/or obesity; e.g. by decreasing food intake, suppressing appetite, inducing satiety, reducing body weight; treating or preventing binge eating disorder, food cravings, bulimia nervosa and/or obesity induced by medication, such as an antipsychotic or a steroid; reducing gastric motility; and/or delaying gastric emptying;
   (iv) the prevention and/or treatment of cardiovascular disease, such as the delaying or reduction of the development of a major adverse cardiovascular event (MACE) selected from the group consisting of cardiovascular death, non-fatal myocardial infarction, non-fatal stroke, revascularisation, hospitalisation for unstable angina pectoris, and hospitalisation for heart failure;
   (v) the prevention and/or treatment of non-alcoholic fatty liver disease (NAFLD) and/or non-alcoholic steatohepatitis (NASH), otherwise known as metabolic dysfunction-associated steatohepatitis (MASH), and/or alcoholic liver disease (ALD);
   (vi) the prevention and/or treatment of chronic kidney disease;
   (vii) the prevention and/or treatment of cognitive disorders including dementia and Alzheimer's disease.
44. The GLP-1 derivative or analogue according to embodiment 43, for use in the prevention or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1c.
45. The GLP-1 derivative or analogue according to embodiment 43, for use in the prevention or delaying diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes.
46. The GLP-1 derivative or analogue according to embodiment 43, for use in the prevention or treatment of certain eating disorders, overweight and/or obesity; e.g. by decreasing food intake, suppressing appetite, inducing satiety, reducing body weight; treating or preventing binge eating disorder, food cravings, bulimia nervosa and/or obesity induced by medication, such as an antipsychotic or a steroid; reducing gastric motility; and/or delaying gastric emptying.
47. The GLP-1 derivative or analogue according to embodiment 43, for use in the prevention or treatment of cardiovascular disease, such as the delaying or reduction of the development of a major adverse cardiovascular event (MACE) selected from the group consisting of cardiovascular death, non-fatal myocardial infarction, non-fatal stroke, revascularisation, hospitalisation for unstable angina pectoris, and hospitalisation for heart failure.
48. The GLP-1 derivative or analogue according to embodiment 43, for use in the prevention or treatment of non-alcoholic fatty liver disease (NAFLD) and/or non-alcoholic steatohepatitis (NASH), otherwise known as metabolic dysfunction-associated steatohepatitis (MASH), and/or alcoholic liver disease (ALD).
49. The GLP-1 derivative or analogue according to embodiment 43, for use in the prevention or treatment of chronic kidney disease:
50. The GLP-1 derivative or analogue according to embodiment 43, for use in the prevention or treatment of cognitive disorders including dementia and Alzheimer's disease.
51. The GLP-1 derivative or analogue according to embodiment 43, for use in the prevention and/or treatment of weight management, obesity and obesity related disorders.
52. The GLP-1 derivative or analogue according to embodiment 43, for use in the prevention and/or treatment of all forms of diabetes, e.g. type 2 diabetes, and diabetes related disorders.
53. Use of a GLP-1 derivative or analogue according to any one of embodiments 1-40 or a composition according to embodiment 41, for the manufacture of a medicament for the treatment of
   (i) Prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1c;
   (ii) Delaying or preventing diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes;
   (iii) the prevention and/or treatment of certain eating disorders, overweight and/or obesity; e.g. by decreasing food intake, suppressing appetite, inducing satiety, reducing body weight; treating or preventing binge eating disorder, food cravings, bulimia nervosa and/or obesity induced by medication, such as an antipsychotic or a steroid; reducing gastric motility; and/or delaying gastric emptying;
   (iv) the prevention and/or treatment of cardiovascular disease, such as the delaying or reduction of the development of a major adverse cardiovascular event (MACE) selected from the group consisting of cardiovascular death, non-fatal myocardial infarction, non-fatal stroke, revascularisation, hospitalisation for unstable angina pectoris, and hospitalisation for heart failure;
   (v) the prevention and/or treatment of non-alcoholic fatty liver disease (NAFLD) and/or non-alcoholic steatohepatitis (NASH), otherwise known as metabolic dysfunction-associated steatohepatitis (MASH), and/or alcoholic liver disease (ALD);
   (vi) the prevention and/or treatment of chronic kidney disease;
   (vii) the prevention and/or treatment of cognitive disorders including dementia and Alzheimer's disease.
54. A method for the treatment or prevention of (i)-(vii), which method comprises administration to a subject in need thereof a therapeutically effective amount of a GLP-1 derivative or analogue according to any one of embodiments 1-40 or the composition according to embodiment 41, wherein (i)-(vii) are:
   (i) Prevention and/or treatment of all forms of diabetes, such as hyperglycemia, type 2 diabetes, impaired glucose tolerance, type 1 diabetes, non-insulin dependent diabetes, MODY (maturity onset diabetes of the young), gestational diabetes, and/or for reduction of HbA1c;
   (ii) Delaying or preventing diabetic disease progression, such as progression in type 2 diabetes, delaying the progression of impaired glucose tolerance (IGT) to insulin requiring type 2 diabetes, delaying or preventing insulin resistance, and/or delaying the progression of non-insulin requiring type 2 diabetes to insulin requiring type 2 diabetes;
   (iii) the prevention and/or treatment of certain eating disorders, overweight and/or obesity; e.g. by decreasing food intake, suppressing appetite, inducing satiety, reducing body weight; treating or preventing binge eating disorder, food cravings, bulimia nervosa and/or obesity induced by medication, such as an antipsychotic or a steroid; reducing gastric motility; and/or delaying gastric emptying;
   (iv) the prevention and/or treatment of cardiovascular disease, such as the delaying or reduction of the development of a major adverse cardiovascular event (MACE) selected from the group consisting of cardiovascular death, non-fatal myocardial infarction, non-fatal stroke, revascularisation, hospitalisation for unstable angina pectoris, and hospitalisation for heart failure;
   (v) the prevention and/or treatment of non-alcoholic fatty liver disease (NAFLD) and/or non-alcoholic steatohepatitis (NASH), otherwise known as metabolic dysfunction-associated steatohepatitis (MASH), and/or alcoholic liver disease (ALD);
   (vi) the prevention and/or treatment of chronic kidney disease;
   (vii) the prevention and/or treatment of cognitive disorders including dementia and Alzheimer's disease.

### EXAMPLES

This experimental part starts with a list of abbreviations and is followed by a section including general methods for synthesising and characterising analogues and derivatives of the invention. Then follows a number of examples which relate to the preparation of specific derivatives of the invention, and at the end a number of examples have been included relating to the activity and properties of these analogues and derivatives (section headed pharmacological methods).

Examples serve to illustrate the invention.

### Materials and Methods

### List of Abbreviations

Ac: acetyl
Aib: alpha-aminoisobutyric acid
AUC: Area Under the Curve
Boc: t-butyloxycarbonyl
DCM: dichloromethane
DIC: diisopropylcarbodiimide
DIPEA: N,N-diisopropylethylamine or Hunig's base
DMF: dimethyl formamide
DODT: 3,6-dioxa-1,8-octanedithiol
DTT: dithiothreitol
EDTA: ethylenediaminetetraacetic acid
ELISA: Enzyme Linked Immuno Sorbent Assay
Fmoc: 9-fluorenylmethyloxycarbonyl
HFIP: 1,1,1,3,3,3-hexafluoro-2-propanol or hexafluoroisopropanol
HOBt: 1-hydroxybenzotriazole
HPLC: High Performance Liquid Chromatography
HSA: Human Serum Albumin
Imp: 3-(imidazol-4-yl)-propionyl
i.v.: intravenously
LCMS or LC-MS: Liquid Chromatography Mass Spectroscopy
MeCN: acetonitrile
Mtt: 4-methyltrityl
NHS: N-hydroxysuccinimide
NMP: N-methyl pyrrolidone
Oxyma Pure®: cyano-hydroxyimino-acetic acid ethyl ester
PK: pharmacokinetic
QTof: Quantitative Time of Flight
s.c.: subcutaneously
SD: Standard Deviation
SEC-HPLC: Size Exclusion High Performance Liquid Chromatography
SEM: Standard Error of Mean
SPPS: solid-phase peptide synthesis
tBu: t-butyl
TFA: trifluoroacetic acid
TIPS: triisopropylsilane
Trt: triphenylmethyl or trityl
UPLC: Ultra Performance Liquid Chromatography

### General Methods of Preparation

### General Methods for Peptide Preparation:

Resins used for the preparation of compounds 1-2 and reference compounds, e.g. pre-loaded Fmoc-Lys(Mtt)-Wang resin (loading 0.30 mmol/g) or other similar pre-loaded and non-pre-loaded resins suitable for SPPS.

The Fmoc-protected amino acid derivatives used in the SPPS comprised, amongst others, the standard recommended building blocks, as well as suitable building blocks to incorporate unnatural amino acids: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, and Fmoc-Val-OH, Fmoc-Aib-OH etc. Optionally, Fmoc-pseudoproline dipeptide building blocks, such as Fmoc-Ser(tBu)-Ser (ψ^{Me,Me}pro)-OH or Fmoc-Val-Ser (ψ^{Me,Me}pro)-OH, could be employed in the SPPS. For the N-terminal amino acid, suitable building blocks were used, e.g. 3-(1-trityl-imidazol-4-yl)propanoic acid (DeaminoHis(Trt)-OH), Boc-His(Trt)-OH, Boc-Tyr(tBu)-OH. The Fmoc amino acids as well as the building blocks used for instalment of the N-terminal residue were supplied from e.g. AAPPTEC, Apigenex, Anaspec, Bachem, ChemImpex, Iris Biotech, Midwest Biotech, Gyros Protein Technologies or Novabiochem. The pseudoproline building blocks were supplied from e.g. technocomm and Iris Biotech. Where nothing else is specified the natural L-form of the amino acids are used. The Lys(mtt) residue was used to allow orthogonal unmasking of the epsilon amine function. The selective Lys(mtt) deprotection was accomplished using a suitable orthogonal deprotecting agent/mix, such as HFIP in DCM. After this the side chain was installed either stepwise with SPPS or by coupling the epsilon amino functionality directly to the activated ester (e.g. NHS ester) sidechain building block.

In case of stepwise side chain attachment using SPPS, the following suitably protected building blocks such as, but not limited to, Fmoc-8-amino-3,6-dioxaoctanoic acid (Fmoc-OEG-OH), Fmoc-Glu-OtBu and octadecanedioic acid mono-tert-butyl ester. The operations stated below were performed within a 50-450 μmol synthesis scale range.

### SPPS of the Peptidyl Backbone:

SPPS was performed using Fmoc based chemistry on a SymphonyX Solid Phase Peptide Synthesizer from Protein Technologies (Tucson, AZ 85714 U.S.A.). Fmoc-deprotection was achieved with e.g. 20% piperidine in DMF, containing e.g. 0-0.3 M Oxyma Pure®. Peptide couplings were performed using DIC/Oxyma Pure®. Amino acid/Oxyma Pure® solutions (e.g. at 0.3 M/0.3 M in DMF at a molar excess of 3-12 fold) were added to the resin followed by the same molar equivalent of DIC (e.g. as a 0.6-1.5M solution in DMF) and collidine (e.g. 1.5M in DMF). The step-wise assembly was done using the following steps: 1) pre-swelling of the preloaded resin with DMF; 2) Fmoc-deprotection by the use of 20% piperidine in DMF containing between 0 and 0.2M Oxyma Pure® for 1-5 treatments of 5-30 min each; 3) washes with DMF to remove traces of piperidine; 4) coupling of Fmoc-amino acid with 3-12 eq. of Fmoc-amino acid as a 0.3M solution in 0.3M Oxyma Pure® in DMF mixed with an equimolar volume of DIC and collidine for 1-12 hours. In the case of sterically hindered amino acids, this coupling step was repeated once or twice; 5) washes with DMF to remove excess reagents. 6) (optional) capping step using a suitable acetic anhydride based capping mixture, e.g. a 3/1 mix of 1.0 M acetic anhydride in DMF and 1.5M collidine in DMF (0.75 M Ac₂O/0.3 M collidine in DMF) for 20 min. Steps 2-5 or, in case a capping step was used, steps 2-6, were repeated using the appropriate Fmoc-amino acid, pseudoproline, or building block suitable for instalment of the N-terminal residue, until the desired length and sequence of the peptide backbone was obtained. 7) final wash with DCM at the completion of the assembly which made the resin ready for attachment of a modifying group on lysine side chain.

### Deprotection of the Lys(Mtt) and Attachment of the Side Chains to Resin Bound Protected Peptide Backbone:

The N-epsilon-lysine Mtt protection group was removed by washing the resin with a suitable orthogonally deprotection mixture, such as HFIP/DCM/TIPS (22.5/75/2.5), in multiple cycles (e.g. 1×10 min and 2×20 min) before washing with DCM (3×) and DMF (4×).

Sidechain installation at the free epsilon amino moiety of the lysine residue was performed either manually, or on a solid phase peptide synthesizer, such as the SymphonyX Solid Phase Peptide Synthesizer from Protein Technologies (Tucson, AZ 85714 U.S.A.). As described in this method's section “SPPS of the peptidyl backbone” using stepwise addition of building blocks such as Fmoc-8-amino-3,6-dioxaoctanoic acid (Fmoc-OEG-OH), Fmoc-Glu-OtBu (Fmoc-gGlu(tBu)-OH). Introduction of the fatty acid moiety was achieved using peptide coupling conditions using a suitable building block, such as octadecanedioic acid mono-tert-butyl ester for a prolonged time (e.g. 2×6 hours).

Alternatively, the side chain moiety could be installed onto the peptidyl backbone resin in a single step, directly after removal of the Mtt group, as described in the first part of this section, by using a suitable side chain building block equipped with an activated ester, such as NHS.

### Cleavage of Resin Bound Peptide and Purification:

After synthesis the resin was washed with DCM (4×), and the peptidyl resin subjected to a suitable TFA based cleavage mixture, such as TFA/TIPS/DTT/H₂O (90/4/3/3, 20 ml at a 0.15 mmol scale). The cleavage reactions would typically be carried out at room temperature and a duration of 1-3 hours (e.g. 2.5 hours). The cleavage reaction was followed by a filtration (using a suitable filter: e.g. P2 sintered glass filter). The crude peptide was precipitated from the filtrate by addition of cold (e.g. 5° C.) diethyl ether (e.g. 40 ml diethyl ether per 10 ml filtrate). The precipitated was further washed with cold diethyl ether and after a short period of drying (e.g. 1 h) dissolved in a suitable aqueous mixture, e.g. a mixture of water, acetic acid and acetonitrile (45/45/10), and filtered before purification by reversed-phase preparative HPLC (Waters Deltaprep 4000) using a suitable C18 prep-HPLC column (e.g. select CSH Prep C18, 5 um, OBD, 30×150 mm). Elution was performed with an increasing gradient of MeCN in water containing 0.1% TFA. Relevant fractions were analysed by analytical UPLC and LC-MS. Fractions containing the pure target peptide were pooled and freeze dried.

Optionally, or when further purification was necessary, the crude peptide or lyophilized peptide TFA salt isolated as described above was dissolved in a neutral (e.g. pH 7-8) aqueous buffer based on common buffer salts such as, but not limited to, sodium hydrogen phosphate and purified with reversed-phase preparative HPLC (Waters Deltaprep 4000) on a C18 column. Elution was performed with an increasing gradient of MeCN in aqueous ammonium bicarbonate (0-10 g/l). Relevant fractions were analysed by analytical UPLC. Fractions containing the pure target peptide were pooled and freeze-dried.

Optionally the peptide TFA salt was converted to its sodium salt by methods known in the art, such as size exclusion chromatography, or reversed phase chromatography methods e.g. C18 silica cartridges or RP-HPLC methods.

### General Methods for LCMS Characterization

LCMS was performed on a set up consisting of Waters Acquity UPLC H Class system and Waters Xevo G2-XS QTof. Eluents: A: 0.1% formic acid in MQ water; B: 0.1% formic acid in MeCN.

The analysis was performed with a column temperature of 60° C. by injecting an appropriate volume (e.g. 1.0 ul) of the sample onto the column which was eluted with a gradient of A and B. The UPLC conditions, detector settings, and mass spectrometer settings were as follows. Column: Waters Acquity BEH, C-18, 1.7 μm, 2.1 mm×50 mm. Gradient: Linear 5%-95% B during 4.0 min at 0.4 ml/min. Detection: Ionisation method: ES, Scanning range: 50-4000 amu, Operating mode: MS resolution mode, positive/ne: positive mode, Voltage: Capillary 3.00 kV, Sample cone 40 V, Source 80 V.
Temperature: Source 150° C., Desolvation 350° C., Scan time 0.500 s, Interscan delay: 0.014 s.

### Example 1: Synthesis of Compounds

The derivatives of the invention and reference compounds were synthesised according to the general methods of preparation as described above.

### Compound 1

### N{Epsilon-37}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[Imp7,Trp8,Glu22,Arg26,Arg34,Lys37]-GLP-1(7-37) (SEQ ID NO: 1)

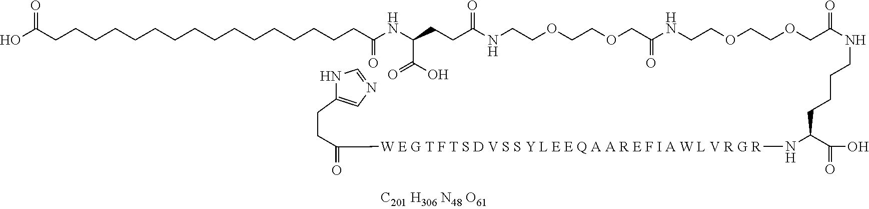

Molecular weight (average) calculated: 4370.87 g/mol

mono isotopic mass: 4368.23 g/mol

LCMS: found (M+4H)4+1093.07 (mono isotopic)

### Compound 2

### N{Epsilon-37}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[Imp7,Gly8,Glu22,Arg26,Arg34,Lys37]-GLP-1(7-37) (SEQ ID NO: 2)

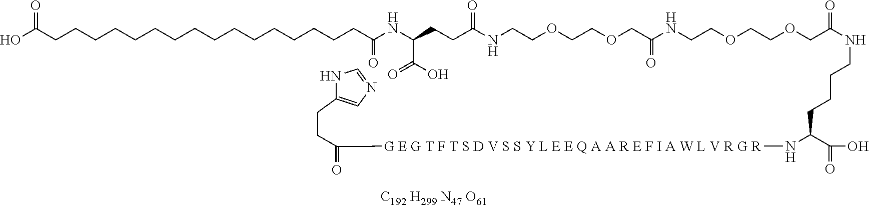

Molecular weight (average) calculated: 4241.71 g/mol

mono isotopic mass: 4239.17 Da

LCMS: found (M+4H)4+1060.79 (mono isotopic)

### Compound 3 (Reference)

### N{Epsilon-37}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[Imp7,Aib8,Glu22,Arg26,Arg34,Lys37]-GLP-1(7-37) (SEQ ID NO: 3)

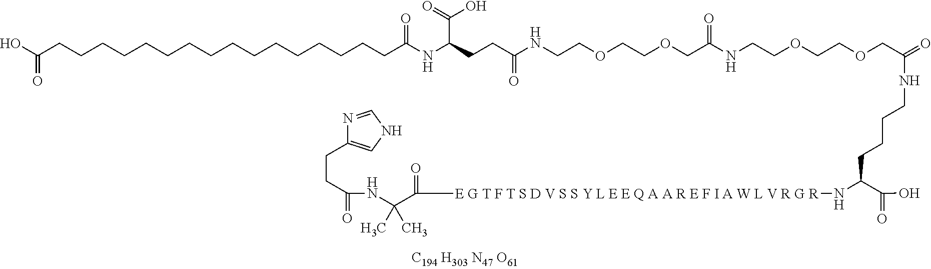

Molecular weight (average) calculated: 4269.76 g/mol

mono isotopic mass: 4267.21 Da

LCMS: found (M+4H)4+1067.80 (mono isotopic)

### Compound 4 (Reference, See WO2009/030738 Ex. 29)

### N{Epsilon-37}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[Imp7,Glu22,Arg26,Arg34,Lys37]-GLP-1(7-37) (SEQ ID NO: 4)

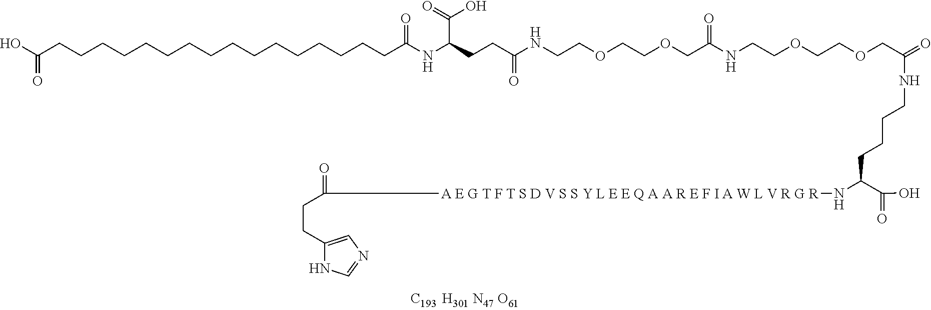

Molecular weight (average) calculated: 4255.73 g/mol

mono isotopic mass: 4253.19 Da

LCMS: found (M+4H)4+1064.30 (mono isotopic)

### Compound 5 (Reference)

### N{Epsilon-37}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[Trp8,Glu22,Arg26,Arg34,Lys37]-GLP-1(7-37) (SEQ ID NO: 5)

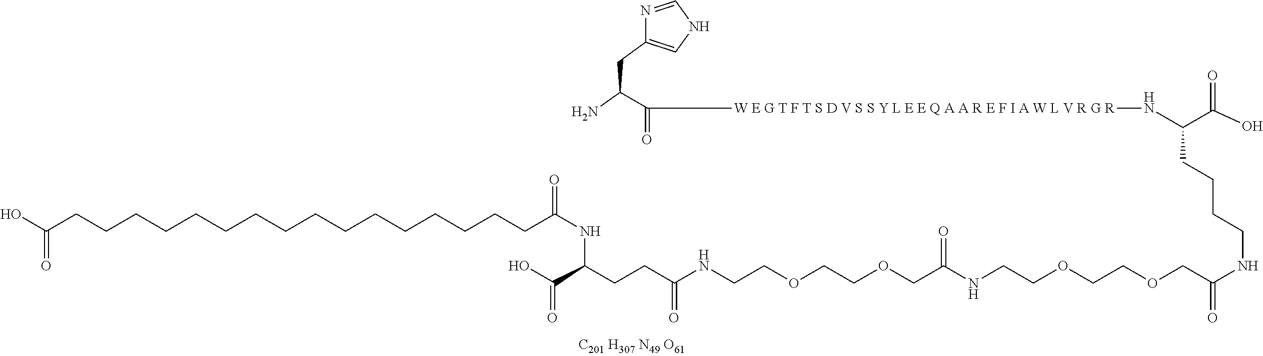

Molecular weight (average) calculated: 4385.88 g/mol

mono isotopic mass: 4383.24 Da

LCMS: found (M+4H)4+1096.81 (mono isotopic)

### Compound 6 (Reference)

### N{Epsilon-37}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[Gly8,Glu22,Arg26,Arg34,Lys37]-GLP-1(7-37) (SEQ ID NO: 6)

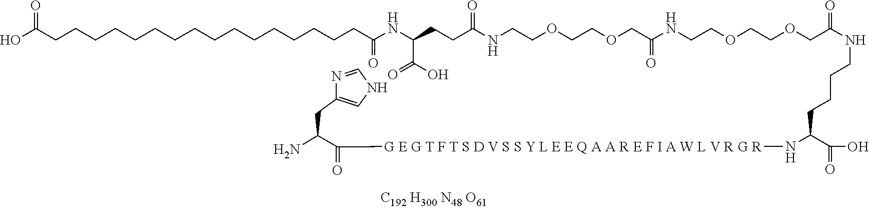

Molecular weight (average) calculated: 4256.72 g/mol

mono isotopic mass: 4254.18 Da

LCMS: found (M+4H)4+1064.54 (mono isotopic)

### Compound 7 (Reference, See WO2009/030738 Ex. 27)

### N{Epsilon-37}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[Aib8,Glu22,Arg26,Arg34,Lys37]-GLP-1(7-37) (SEQ ID NO: 7)

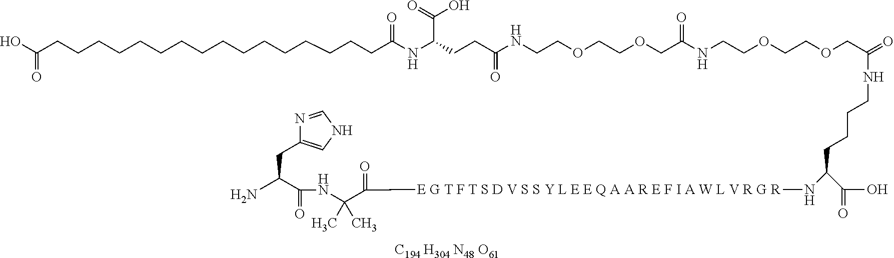

Molecular weight (average) calculated: 4284.77 g/mol

mono isotopic mass: 4282.22 Da

LCMS: found (M+4H)4+1071.55 (mono isotopic)

### Compound 8 (Reference)

### N{Epsilon-37}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[Glu22,Arg26,Arg34,Lys37]-GLP-1(7-37) (SEQ ID NO: 8)

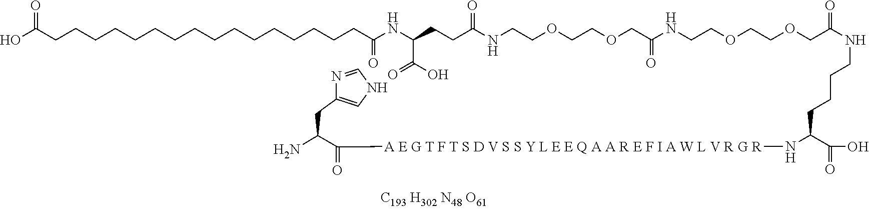

Molecular weight (average) calculated: 4270.75 g/mol

mono isotopic mass: 4268.20 Da

LCMS: found (M+4H)4+1068.06 (mono isotopic)

### Compound 9 (Reference)

### [Imp7,Trp8,Glu22,Arg26,Arg34]-GLP-1(7-37) (SEQ ID NO: 9)

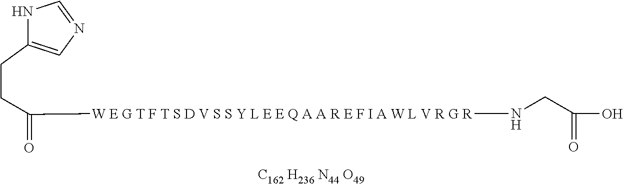

Molecular weight (average) calculated: 3583.87 g/mol

mono isotopic mass: 3581.73 Da

LCMS: found (M+4H)4+896.44 (mono isotopic)

### Compound 10 (Reference)

### [Aib8,Arg34]-GLP-1(7-37) (SEQ ID NO: 10)

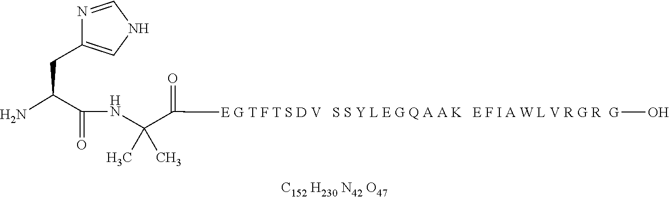

Molecular weight (average) calculated: 3397.71 g/mol

mono isotopic mass: 3395.69 Da

LCMS: found (M+4H)4+849.93 (mono isotopic)

### Compound 11 (Reference)

### hGLP-1(7-37) (SEQ ID NO: 11)

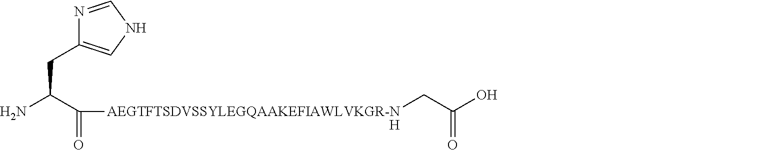

### Compound 12 (Reference)

Semaglutide (SEQ ID NO: 12) (see e.g. J. Med. Chem. 2015, 58, 18, 7370-7380); may alternatively, be prepared as described in WO2006/097537, Example 4.

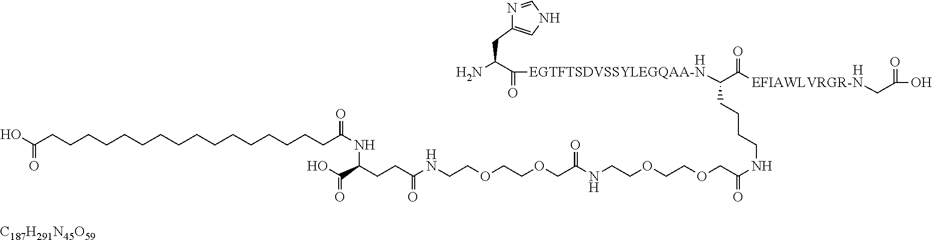

Molecular weight (average) calculated: 4113.58 g/mol

mono isotopic mass: 4111.12 Da

LCMS: found (M+4H)4+1028.79 (mono isotopic)

### Compound 13 (Reference)

### N{Epsilon-26}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[Imp7,Aib8,Arg34]-GLP-1(7-37) (SEQ ID NO: 13)

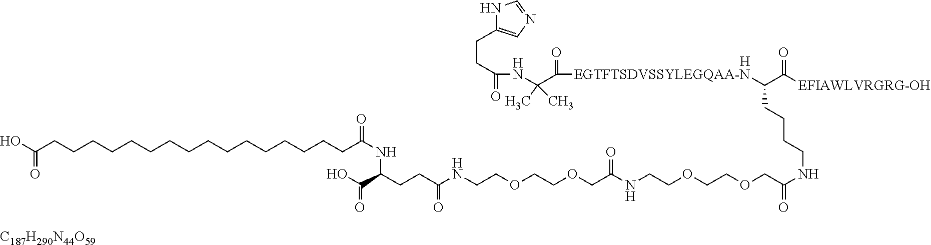

Molecular weight (average) calculated: 4098.56 g/mol

mono isotopic mass: 4096.10 Da

LCMS: found (M+4H)4+1025.03 (mono isotopic)

### COMPOUND 14 (Reference, See WO2017/149070 Ex. 2)

### N{Epsilon-26}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[Trp8,Arg34]-GLP-1(7-37) (SEQ ID NO: 14)

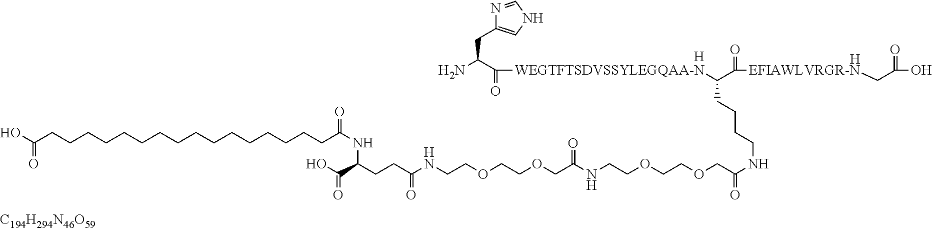

Molecular weight (average) calculated: 4214.68 g/mol

mono isotopic mass: 4212.14 Da

LCMS: found (M+4H)4+1054.03 (mono isotopic)

### Compound 15 (Reference)

### N{Epsilon-26}-[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]-[Imp7,Trp8,Arg34]-GLP-1(7-37) (SEQ ID NO: 15)

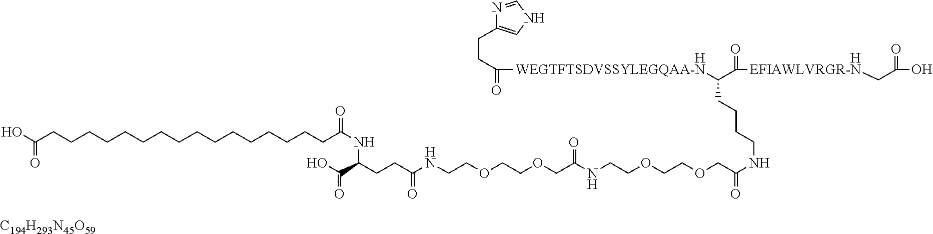

Molecular weight (average) calculated: 4199.67 g/mol

mono isotopic mass: 4197.13 Da

LCMS: found (M+4H)4+1050.29 (mono isotopic)

### Compound 16 (Reference)

### Ecnoglutide (SEQ ID NO: 16) (See e.g. Mol Metab, 2023, 75, 101762)

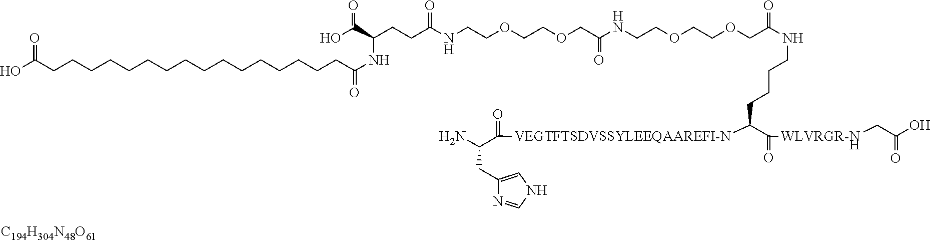

Molecular weight (average) calculated: 4284.77 g/mol

mono isotopic mass: 4282.22 Da

LCMS: found (M+4H)4+1071.58 (mono isotopic)

### Compound 17 (Reference, WO2019/211451 Ex.1 Comp 31)

### N{1}-acetyl,N{Epsilon-24}-[(4S)-4-carboxy-4-[[(4S)-4-carboxy-4-[[(4S)-4-carboxy-4-[[4-[(19-carboxynonadecanoylamino)methyl]cyclohexanecarbonyl]-amino]butanoyl]amino]butanoyl]amino]butanoyl]-[D-Tyr1,Aib2,Nle14,Arg18,Lys24,Pro31]-hGIP(1-31) amide (SEQ ID NO: 17)

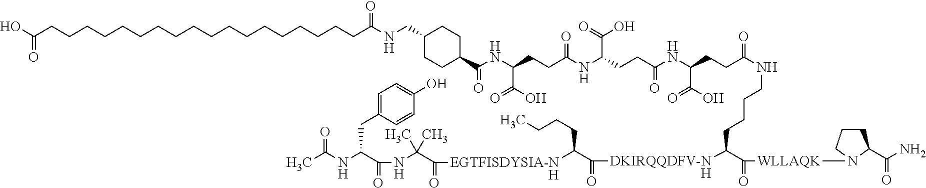

Molecular weight (average) calculated: 4551.23 g/mol

LCMS: found (M+4H)4+1138.12

### Compound 18 (Reference, See WO2017/149070 Ex. 10)

### N{Epsilon-37}-[(2S)-2,6-bis[[2-[2-[2-[[2-[2-[2-[[2-[2-[2-[[2-[2-[2-[[2-[2-[2-[[2-[2-[2-[[(4S)-4-carboxy-4-[[4-[(19-carboxynonadecanoylamino)methyl]cyclohexanecarbonyl]amino]butanoyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]ethoxy]ethoxy]acetyl]amino]hexanoyl]-Trp8,Glu22, Arg26, Arg34, Lys37]-GLP-1(7-37) (SEQ ID NO: 22)

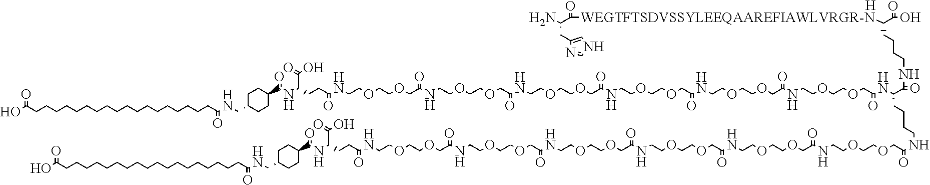

Molecular weight (average) calculated: 6725.67 g/mol

Mono isotopic mass: 6721.62 Da

LCMS: found (M+5H)5+1345.34 (mono isotopic)

### Example 2: In Vitro Potency (hGLP-1R cAMP Generation)

The purpose of these studies is to determine the absolute and relative potency of Compound 1 and Semaglutide to induce cAMP accumulation by activation of human GLP-1R. This is assessed by a cell-based assay, where engineered BHK-21 cells respond to cAMP accumulation by expressing a firefly luciferase reporter gene. This luciferase produces a luminescent readout proportional to the amount of cAMP in the cell upon addition of a cell permeable substrate. Cells are exposed to serial dilutions of relevant test compound, after which ‘potency’ is assessed by calculating an EC₅₀ value from a concentration response curve, corresponding to the concentration of test compound required to give half the maximal response possible in the assay.

Cell culture. The cell line used for this assay (BHK-FCW467-12A KZ10-1 hGLP-1R CRE-luc) is derived from a BHK-21 parental cell line stably transfected with hGLP-1R and cAMP response element (CRE)-luciferase reporter gene. Cells were grown in DMEM (Gibco) containing 10% heat inactivated fetal bovine serum (FBS, Gibco), 1% Penicillin/Streptomycin (Gibco), 1× Sodium Pyruvate (Gibco), 0.5 mg/ml G418 (Gibco) and 240 nM Methotrexate (Pfizer). Cells were maintained at 37° C. in a humidified incubator. Cells were harvested and frozen down at −80° C. in assay-ready aliquots in a medium containing 20% FBS and 10% DMSO for cryopreservation. After freezing cells were moved to Liquid Nitrogen for longer-term storage.

Luciferase assay. Test compounds were serially diluted to 3× final concentration in assay buffer (DMEM w/o phenol red, Glutamax, HEPES 1M, Pluronic F-68, 10% Ovalbumin (OVA) using a Biomek liquid handler. 10 μl of assay buffer was added to each well of a 384w plate, containing either 0 or 3% Human serum albumin (HSA), followed by 10 μl of diluted compound and finally 10 μl of thawed, once-washed, assay-ready cells at a final concentration of 1500 cells/well. The final concentration of HSA was either 0 or 1%. Plates were briefly spun down and incubated for 3-hours at 37° C. in a humidified incubator. After incubation, cells were equilibrated at room temperature for 5 minutes before 30 μl/well of Steadylite Plus (luciferase substrate) was added. Plates were covered in foil and shaken at room temperature for 30 minutes. Plates were then sealed with TopSeal film and signal read on a microplate reader capable of detecting luminescence (Biotek Synergy2). Potency (EC₅₀) values are derived from 3 parameter concentration response curves fit in Graphpad prism. Results are shown in Table 1 below:

TABLE 1
in vitro characterization of cAMP potency at hGLP-1R.

| | 0% HSA | | 1% HSA | | |
|---|---|---|---|---|---|
| | EC₅₀/pM | EC₅₀ | EC₅₀/pM | EC₅₀ | |
| Compound | Absolute ± SD | Relative* ± SD | Absolute ± SD | Relative* ± SD | n |
| Compound 1 | 17.3 ± 3.5 | 2.9 ± 0.5 | 601 ± 89 | 4.2 ± 0.1 | 2 |
| Semaglutide | 6.2 ± 0.2 | 1.0 ± 0.0 | 134.5 ± 3.5 | 1.0 ± 0.0 | 2 |

SD: standard deviation;
*relative to semaglutide

Data in Table 1 shows the absolute and relative potencies of Compound 1 of the invention compared to known GLP-1 agonists semaglutide. It is seen that all compounds activate the GLP-1R and induce a cAMP response.

### Example 3: In Vitro Potency (HGIPR Camp Generation)

The purpose of these studies is to determine the relative potency of Compound 1, compared to a known human GIP agonist (compound 17) to induce cAMP accumulation by activation of human GIP and show that Compound 1 is a selective GLP-1 receptor agonist.

Methods: Assay is performed as described above for hGLP-1R in Example 2, but using cells expressing human GIPR, instead of hGLP-1R. Results are shown in Table 2 below:

TABLE 2
in vitro characterization of cAMP potency at hGIPR.

| | 0% HSA | | 1% HSA | | |
|---|---|---|---|---|---|
| | EC₅₀/pM | EC₅₀ | EC₅₀/pM | EC₅₀ | |
| Compound | Absolute ± SD | Relative^{#} ± SD | Absolute ± SD | Relative^{#} ± SD | n |
| Compound 17 | 3.3 ± 0.5 | 1.0 ± 0.0 | 107.0 ± 21.1 | 1.0 ± 0.0 | 2 |
| Compound 1 | >10,000 | >1,000 | >10,000 | >1,000 | 2 |

SD: standard deviation;
^{#}relative to known hGIP agonist Compound 17.

The data shows that Compound 1 does not activate the hGIPR and is selective at the GLP-1 receptor over the GIP receptor.

### Example 4: In Vitro hGLP-1R Beta-Arrestin 1+2

The purpose of these studies is to determine the ability of Compound 1, Compound 2, Semaglutide, various other GLP-1 receptor agonists, and Ecnoglutide to induce β-arrestin1 or β-arrestin2 recruitment to GLP-1R in HEK293 cells. This is done in an engineered cell line expressing a split Nano Luciferase, the Large BiT (LgBiT, 18 kDa) component being fused to the GLP-1R C-terminus and the Small BIT (SmBiT, 11 amino acids) being fused to one of the 2 isoforms of β-arrestin. When GLP-1R is activated, it causes translocation of β-arrestin to the receptor, the subsequent association of LgBiT and SmBiT forms a functional luciferase whose activity can be detected by addition of a substrate.

Cell culture: Cells were grown in DMEM (Gibco) containing 10% heat inactivated fetal bovine serum (FBS, Gibco), 1% Penicillin/Streptomycin (Gibco), 1× Sodium Pyruvate (Gibco), 0.5 mg/ml G418 (Gibco) and 240 nM Methotrexate (Pfizer). Cells were maintained at 37 degrees in a humidified incubator.

Experimental procedure: Upon reaching approx. 80% confluency, cells were harvested using trypsin and resuspended in standard cell medium to a concentration of 250,000 cells/ml. 100 μl of cell suspension was added per well to a PDL coated, white, opaque, 96 well microplate to achieve a final density of 25,000 cells/well. Plates were incubated for 2 days at 37° C. in a humidified incubator. On the day of the experiment, plates were washed 1× with PBS before addition of 70 ul of DMEM without phenol red+1% Ovalbumin (Assay buffer) and incubated for 1 hr at 37° C. Serial dilution of compounds at 10× final concentration were made in assay buffer. Nano-Glo substrate (Promega) was diluted 100× in assay buffer and 20 ul was added to each well, before placing in a microplate reader pre-heated to 37° C. 10 minutes to equilibrate (BMG Clariostar). Luminescence was read across the plate in a kinetic mode, reading a value per well every 115 s. After a baseline reading of 5 cycles, the experiment was paused and 10 ul of compounds added in duplicate using a multichannel pipette. Luminescence was read for a further 15 cycles. For concentration response curves, area under the curve was calculated for each well, after normalization to individual baseline luminescence and subtraction of an average vehicle-treated control. Eₘₐₓ, determined as the top of the fitted concentration response curve, was normalized to the Eₘₐₓ of hGLP-1(7-37), which was ran on every experimental plate as a common reference compound.

TABLE 3
in vitro characterization of β-arrestin1 and β-arrestin2 signaling at GLP-1R.

| | β-arrestin 1 | | β-arrestin2 | | |
|---|---|---|---|---|---|
| | EC₅₀/nM | Eₘₐₓ/% | EC₅₀/nM | Eₘₐₓ/% | |
| Compound | Absolute ± SD | Relative^{&} ± SD | Absolute ± SD | Relative^{&} ± SD | n |

Analogues with no substituent

| | | | | | |
|---|---|---|---|---|---|
| hGLP-1(7-37) | 24.8 ± 3.6 | 100 | 24.0 ± 1.5 | 100 | 3 |
| Compound 9 | 51.3 ± 21.2 | 48 ± 3 | 29.9 ± 0.9 | 56 ± 1 | 2 |
| Compound 10 | 6.1 | 87 | 6.2 | 95 | 1 |

Derivatives with substituent at 37K varying only in positions 7 and 8

| | | | | | |
|---|---|---|---|---|---|
| Compound 1 | 41.9 ± 12.0 | 26 ± 2 | 30.3 ± 5.2 | 38 ± 3 | 3 |
| Compound 2 | 76.2 ± 19.2 | 30 ± 2 | 47.3 ± 4.6 | 43 ± 1 | 2 |
| Compound 3 | 21.5 ± 2.4 | 88 ± 1 | 22.2 ± 2.1 | 83 ± 3 | 2 |
| Compound 4 | 28.8 ± 3.3 | 76 ± 0 | 27.4 ± 0.5 | 76 ± 1 | 2 |
| Compound 5 | 24.5 ± 1.2 | 89 ± 1 | 23.2 ± 0.2 | 83 ± 2 | 2 |
| Compound 6 | 21.5 | 87 | 22.7 | 84 | 1 |
| Compound 7 | 14.2 | 92 | 17.4 | 97 | 1 |
| Compound 8 | 17.6 | 101 | 26 | 100 | 1 |
| Compound 18 | 400 | 103 | 334.1 | 96 | 1 |

Derivatives with substituent at 26K varying only in positions 7 and 8

| | | | | | |
|---|---|---|---|---|---|
| Semaglutide | 27.39 ± 5.52 | 106 ± 5 | 23.99 ± 8.10 | 108 ± 6 | 3 |
| Compound 13 | 71.3 | 86 | 47.2 | 91 | 1 |
| Compound 14 | 39.0 | 92 | 33.7 | 87 | 1 |
| Compound 15 | 1356 | 74 | 495 | 70 | 1 |

Reference to biased agonist

| | | | | | |
|---|---|---|---|---|---|
| Ecnoglutide | 26.8 ± 8.3 | 79 ± 5 | 25.5 ± 10.3 | 80 ± 3 | 3 |

SD: standard deviation;
^{&}relative to hGLP-(7-37)

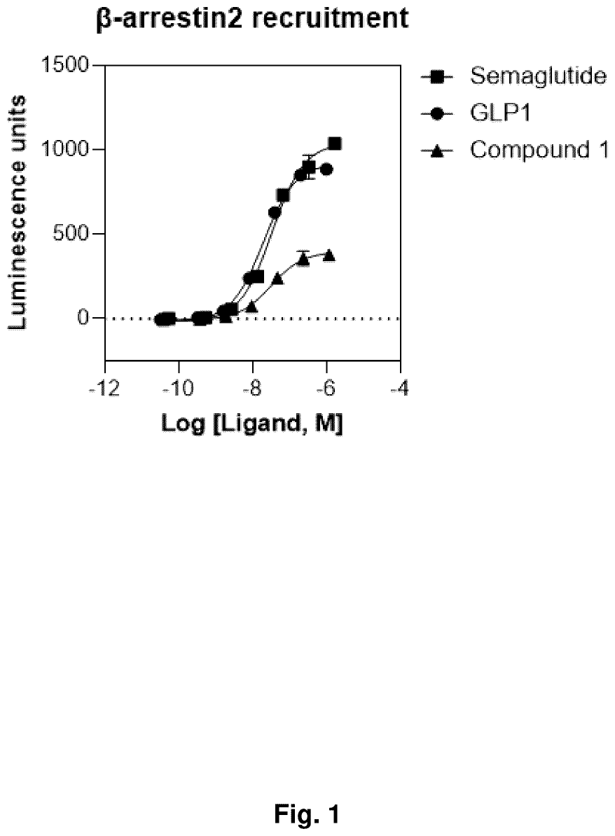

The data demonstrates that Compounds 1 and 2 of the invention have significantly reduced efficacy (Eₘₐₓ) in the beta-arrestin assay, for both isoform 1 and 2 over all other tested compounds even compared to e.g. Compounds 5 and 6 that only differ from Compounds 1 and 2 in having His vs. Imp at the N-terminal or Compound 18 differing from Compound 1 in having His at the N-terminal and a different substituent at the C-terminal Lys. Concentration response curves for Compound 1 compared to semaglutide (Compound 12) and hGLP-(7-37) (Compound 11) is shown in FIG. 1.

In addition, it is seen that Compound 18 (differing in His at N-terminal and different substituent at C-terminal Lys) and Compound 15 (has Imp at N-terminal and differs mainly in having the substituent in a different position) have significantly worse potency at beta-arrestin recruitment (EC₅₀) than e.g. Compounds 1 and 2 of the present invention.

### Example 5: Pharmacokinetic Study in Minipigs

The purpose of this study is to determine the half-life in vivo of compound 1 and compare with semaglutide after i.v. administration to minipigs, i.e. the prolongation of their time in the body and thereby their time of action. This is done in a pharmacokinetic (PK) study in Göttingen Minipigs, where the terminal half-life of the compound in question is determined. By terminal half-life is generally meant the period of time it takes to halve a certain plasma concentration, measured after the initial distribution phase.

Animals and housing. Female Göttingen Minipigs were obtained from Ellegaard Minipigs, Dalmose Denmark. The minipigs were housed individually and were fed restrictedly twice daily with Altromin 9033 (Brogaarden ApS, Lynge, Denmark). After minimum 2 weeks of acclimatization two permanent central venous catheters were implanted during general anaesthesia in either the caudal or cranial caval vein in each animal. The animals were allowed approximately 1 week recovery after the surgery and were then used for repeated pharmacokinetic studies with a suitable wash-out period between successive dosing episodes.

Body Weight. The animals were weighed weekly and either on the dosing day or the day before dosing to decide the correct dosing volume. The minipigs weighed approx. 25 kg on the dosing days.

Formulation: The following formulations were used in these PK studies: compound 1 was formulated in: 8 mM phosphate, 250 mM glycerol, 0.007% polysorbate 20, pH 7.4. and semaglutide was formulated in: 8 mM phosphate; 184 mM propylene glycol; 58 mM phenol. pH 7.4.

Administration of peptides and dosing solutions. There were no food restrictions during the study and the animals had ad libitum access to water during the whole study period. Intravenous injections of the compounds were given through the short central-venous catheter, which was flushed with minimum 10 ml of sterile saline post administration. Compound 1 was dosed at 1.5 nmol/kg using the dose volume of 0.1 ml/kg and semaglutide was dosed at 2 nmol/kg using the dose volume of 0.046 ml/kg (n=3). Blood samples of 1-1.3 ml were taken through the long central-venous catheter at the following time points in relation to the dosing: Predose, 0.083, 0.25, 0.5, 1, 1.5, 2, 3, 4, 6, 8, 10, 24, 30, 48, 72, 96, 144, 168, 192, 216, 240, 264, 312, 336, 360, 384, 408, 432 and 480 h (3 h time point only for semaglutide and 336-480 h time points only for compound 1). After each blood sample the catheter was flushed with minimum 5-10 ml of sterile 0.9% NaCl containing 10 IE/ml heparin. Aseptic technique was demanded to avoid bacterial growth in the catheter that otherwise increases the risk of clot formation in the catheter. Immediately after sampling, the blood was transferred to test tubes containing K₃EDTA buffer (8 mM). The tubes were kept on wet ice until centrifugation for 10 min at 4° C. and approx. 2000 g within ½ h after sampling. Afterwards, plasma (min. 200 μl) was transferred to Micronic tubes on dry ice and kept at −20° C. until analysis. The plasma samples were analysed by LC/MS as described below.

### Quantitative Assay for Plasma Samples.

Plasma concentrations of Compound 1 were assayed by plasma protein precipitation and analysed by turboflow liquid chromatography mass spectrometry (TF-LC-MS). Calibrators were prepared by spiking blank minipig plasma with Compound 1 in the range from 0.2 to 200 nM. Calibrators, quality controls (QCs), plasma blanks, and study samples were prepared for TF-LC-MS by protein precipitation by adding four volumes of ethanol, containing 20 nM of internal standard, to one volume of sample. After the addition of the precipitation reagent, the mixture was agitated, followed by centrifugation. After centrifugation, one volume of supernatant was mixed with one volume of water (with 1% v/v formic acid).

The mixture was analysed by TF-LC-MS using a Cyclone turboflow column (0.5×50 mm, ThermoFisher Scientific) and a XBridge Peptide BEH C18 3.5 um 300 Å analytical column (50×2.1 mm and obtained from Waters). Gradient elution was conducted using mobile phase A (consisting of milli-Q water with 1% formic acid and 5% methanol/acetonitrile (50/50)) and mobile phase B (consisting of methanol/acetonitrile (50/50) with 1% formic acid and 5% milli-Q water). The mass spectrometer was operated in positive ionization mode. A TSQ Altis mass spectrometer (ThermoFisher Scientific) was used as detector in selected reaction monitoring (SRM) mode (Compound 1: m/z 1093.55→1354.72 and ISTD: m/z 1100.38→1363.97). Linear calibration curves (weighed 1/×2) were used for calculating the concentration in the plasma samples. Quality control samples were included. The acceptance criteria were as follows: The deviation between nominal and calculated concentration in the calibrators and quality control samples was below 15% for 75% of all standards and QC samples.

Plasma concentrations of semaglutide were assayed by plasma protein precipitation and analysed by turboflow liquid chromatography mass spectrometry (TF-LC-MS). Calibrators were prepared by spiking blank minipig plasma with semaglutide in from 0.5 to 2000 nM. Calibrators, quality controls (QCs), plasma blanks, and study samples were prepared for TF-LC-MS by protein precipitation, by addition of four volumes of methanol, containing 20 nM of internal standard, to one volume of plasma. After the addition of the precipitation reagent, the mixture was agitated, followed by centrifugation. After centrifugation, one volume of supernatant was mixed with one volume of water (containing 1% v/v formic acid).

The mixture was analysed by TF-LC-MS using a Cyclone turboflow column (0.5×50 mm, Thermo Fisher Scientific) and an XBridge Protein BEH C4 analytical column (50×2.1 mm, 3.5 μm, 300 Å from Waters). Gradient elution was conducted using mobile phase A (consisting of milli-Q water with 1% formic acid and 5% methanol/acetonitrile (50/50)) and mobile phase B (consisting of methanol/acetonitrile (50/50) with 1% formic acid and 5% milli-Q water). The mass spectrometer was operated in positive ionization mode. A Q-Exactive Plus mass spectrometer (Thermo Fisher Scientific) was used as detector in selected ion monitoring mode (semaglutide: 1028-1032 m/z and ISTD: 1295-1305 m/z). A linear calibration curves with 1/×2 weighting, was used for calculating the concentration in the plasma samples. The acceptance criteria were as follows: The deviation between nominal and calculated concentration in the calibrators and quality control samples was below 15% for 75% of all standards and QC samples.

Data management. Individual pharmacokinetic profiles of plasma concentration versus time data were generated and evaluated by non-compartmental pharmacokinetics analysis using Phoenix® WinNonlin® version 8.4 (Certara L.P., Princeton, NJ, USA). The terminal elimination phase was fitted via linear regression with uniform weighting. Nominal sampling times and actual doses were used for the analysis. Terminal half-life is given in Table 4 as the harmonic mean and minimum and maximum values.

### Results

TABLE 4
Terminal half-life (t_{1/2}) as measured
after i.v. administration to minipigs

| | t_{1/2} (hours) | Nominal dose | Dose volume | |
|---|---|---|---|---|
| Compound | (min; max) | (nmol/kg) | (ml/kg) | n |
| Compound 1 | 157 (147-164) | 1.5 | 0.1 | 3 |
| Semaglutide | 69.9 (65.3-73.5) | 2 | 0.046 | 3 |

The above results show that Compound 1 of the present invention has about twice as long a half-life as compared with semaglutide potentially indicating that reduced beta-arrestin activation prolongs the half-life via reduced receptor mediated clearance. The measured half-life in minipigs predicts a half-life in humans sufficient for at least once-weekly administration via liquid injection. Similar PK data after subcutaneous dosing have been generated in mice showing similar relative difference in half-life.

### Example 6: Physical Stability (Fibril Formation; ThT Fluorescence)

The purpose of this study was to assess the physical stability of the compounds of the invention in aqueous solution in a ThT assay as explained below.

### Peptide Quantification by Charged Aerosol Detector (CAD).

CAD was performed on a Thermo Scientific Vanquish UPLC system with a UV-DAD and CAD (Charged Aerosol Detector). The system was fitted with a Waters ACQUITY UPLC, CSH C18 column, 130 Å, 1.7 μm, 2.1 mm×50 mm, column temperature 40° C. UV-detection was at 214 nm. Eluent A: 0.1% v/v TFA in water; eluent B: 0.1% v/v TFA in MeCN. The analysis was performed by injecting an appropriate volume of the sample onto the column which was eluted with a linear gradient of eluents A and B from 0-95% B over 4 min at a constant flowrate of 0.45 ml/min. Runtime: 6 min. Calibration was performed using a standard curve generated by injecting a standard solution of Insulin Aspart, 3.5 mg/ml.

### Sample Preparation.

Freeze dried peptide was dissolved in water and mixed with stock solutions of phosphate and d-sorbitol to give a nominal composition of 3 mg/ml peptide, 8 mM phosphate and 253 mM d-sorbitol. pH was measured and adjusted to pH 7.4 using diluted hydrochloric acid and sodium hydroxide. The samples were sterile filtrated (0.22 μm) to glass vials and stored in the fridge until testing.

### ThT Assay.

Low physical stability of peptides may lead to amyloid fibril formation, which is observed as well-ordered, thread-like macromolecular structures in the sample eventually resulting in gel formation. This has traditionally been tested by visual inspection of the sample. However, that kind of measurement is very subjective and depending on the observer. Therefore, the application of a small molecule indicator probe is much more advantageous. Thioflavin T (ThT) is such a probe and has a distinct fluorescence signature when binding to fibrils [Naiki et al. (1989) Anal. Biochem. 177, 244-249; LeVine (1999) Methods. Enzymol. 09, 274-284].

Thioflavin T was added to the supernatant samples as prepared above using an aqueous ThT stock solution to a final concentration of about 1 μM in the samples. Sample aliquots of 150 μl were placed in a 96 well microtiter plate (Packard OptiPlate™-96, white polystyrene). Three/four replicas of each sample were applied to the plate. The plate was sealed and placed in a fluorescence plate reader (Fluoroskan Ascent FL) and incubated at 37° C. with orbital shaking (900 rpm; 1 mm amplitude). Fluorescence measurements were performed every 20 minutes using excitation through a 444 nm filter and measurement of emission through a 485 nm filter. Between each measurement, the plate was shaken and heated as described above and the assay was terminated after 45 hours. Fluorescence measurements for each microtiter plate well were plotted against time and the lag-time (time until an increase in ThT fluorescence was observed) was estimated. If no increase in ThT fluorescence was observed, the lag-time was set to equal the duration of the assay (45 h). To assess loss of dissolved peptide following 45 h incubation, peptide recovery was measured as the ratio between supernatant peptide concentration after incubation vs. before incubation using the CAD quantification method as given above.

Samples of Compound 1 and semaglutide were prepared as described above and subjected to the ThT test as described above.

### Results

TABLE 5
LagTime and Peptide recovery values

| Compound | LagTime (h) | Peptide Recovery (%) |
|---|---|---|
| Compound 1 | 45 | 113 |
| Semaglutide | 45 | 118 |

Results show no increase in the ThT fluorescence over the duration of the assay (45 h) and no loss of dissolved peptide (Recovery≥100%) suggest high stability of Compound 1 against fibril formation.

### Example 7: Chemical Stability (HMWP Formation; Purity Loss)

The aim of this study was to determine the chemical stability of Compound 1. As a measure of the stability of Compound 1, the formation of HMWP formation (% HMWP) as a function of the time was analysed by size-exclusion chromatography (SEC-MS). Further, the purity loss of Compound 1 was measured by RP-UPLC.

### Incubation.

Samples prepared as “sample preparation” in Example 7 were incubated at 37° C. and aliquots were pulled following two- and four-weeks incubation and compared with corresponding samples pulled prior to incubation. Aliquots were stored at minus 18° C. until measurement of High-Molecular-Weight-Protein (HMWP) and Purity.

### HMWP (High Molecular Weight Protein)

The relative amount of covalent HMWP was assessed by SEC-UPLC with a Waters BEH125 Å, Insulin column (4.6×150 mm, 1.7 μm), isocratic elution (0.3M NaCl, 10 mM NaH₂PO₄ and 5 mM H₃PO₄, 50% (v/v) isopropanol, pH 2.4) with a flow rate of 0.3 ml/min, a column temperature at 50° C. and UV-detection at 215 nm. The total area of peaks eluting prior to the monomeric main peak was termed HMWP (High-Molecular-Weight-Protein) and quantified on a percentage scale relative to the total peptide peak area. To ease comparison, results were normalized by subtracting the amount of HMWP in the corresponding sample pulled prior to incubation. Results are shown in Table 6.

### Purity Loss

The relative peptide purity was assessed by RP-UPLC (Acquity, Waters Corp, Milford, MA, USA) using a BEH C4 column (2.1×150 mm, Waters Corp, Milford, MA, USA) with a linear gradient of acetonitrile in trifluoro acetic acid elution (A: 0.1% TFA, B:0.08% TFA in ACN; 25-50% B in 55 min) at a flow rate of 0.1 ml/min and a column temperature of 60° C. with UV-detection at 215 nm. Purity was calculated as the total area of the monomeric main peak on a percentage scale relative to the total peptide peak area. To ease comparison, results were normalized by subtracting the purity of the sample pulled prior to incubation to give the purity loss below. Results are shown in Table 6.

### Results

TABLE 6
HMWP formation and purity loss at 37° C.

| | HMWP | HMWP | Purity | Purity |
|---|---|---|---|---|
| | Form (%) | Form (%) | Loss (%) | Loss (%) |
| Compound | 2 weeks | 4 weeks | 2 weeks | 4 weeks |
| Compound 1 | 0.31 | 0.50 | 0.38 | 1.2 |
| Semaglutide | 0.33 | 0.51 | 1.7 | 4.3 |

As seen from Table 6, only a minor increase in HMWP formation is seen for both Compound 1 and semaglutide. However, a lower purity loss was seen for Compound 1 as compared to semaglutide.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A GLP-1 derivative comprising:
(SEQ ID NO: 18)
Imp-X₈EGTFTSDVSSYLEEQAAREFIAWLVRGRK;
wherein Imp is 3-(imidazol-4-yl)-propionyl;
wherein X₈ is W or G;
wherein K is chemically modified at the epsilon amino group with: 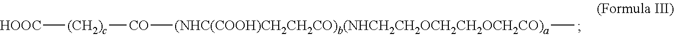
and
wherein a is 1-3, b is 1-2, and c is 14-18;
or a pharmaceutically acceptable salt, amide, or ester thereof.

2. The GLP-1 derivative according to claim 1, wherein X₈ is W.

3. The GLP-1 derivative according to claim 1, wherein X₈ is G.

4. The GLP-1 derivative according to claim 1, wherein a is 2.

5. The GLP-1 derivative according to claim 1, wherein b is 1.

6. The GLP-1 derivative according to claim 1, wherein c is 16.

7. The GLP-1 derivative according to claim 1, wherein the substituent is HOOC—(CH₂)₁₆—CO—(NHC(COOH)CH₂CH₂CO)—(NHCH₂CH₂OCH₂CH₂OCH₂CO)₂— (Formula IIIa).

8. The GLP-1 derivative according to claim 1, wherein the substituent is:

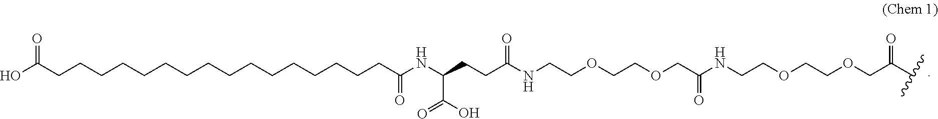 [SMILES: *C(=O)COCCOCCNC(=O)COCCOCCNC(=O)CC[C@H](NC(=O)CCCCCCCCCCCCCCCCC(=O)O)C(=O)O]

9. A GLP-1 derivative consisting essentially of:
(SEQ ID NO: 18)
Imp-X₈EGTFTSDVSSYLEEQAAREFIAWLVRGRK;
wherein Imp is 3-(imidazol-4-yl)-propionyl;
wherein X₈ is W or G;
wherein K is chemically modified at the epsilon amino group with: 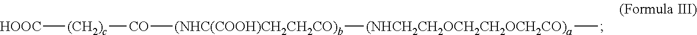
and
wherein a is 1-3, b is 1-2, and c is 14-18;
or a pharmaceutically acceptable salt, amide, or ester thereof.

10. The GLP-1 derivative according to claim 1, wherein the GLP-1 derivative is: 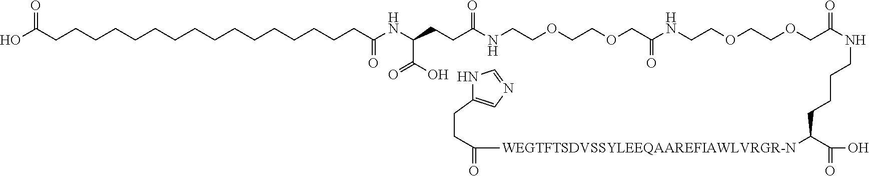
(Compound 1) or a pharmaceutically acceptable salt, amide, or ester thereof.

11. The GLP-1 derivative according to claim 1, wherein the GLP-1 derivative is: 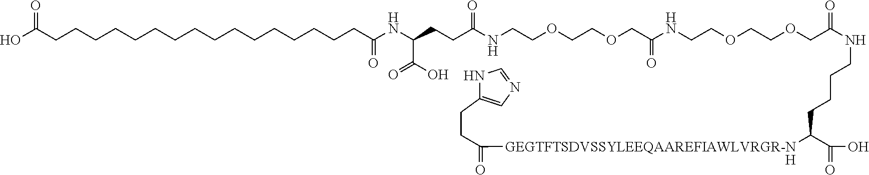
(Compound 2) or a pharmaceutically acceptable salt, amide, or ester thereof.

12. A method of treating overweight, comprising administering the GLP-1 derivative according to claim 1 to a subject in need thereof.

13. The method according to claim 12, wherein the subject is suffering from obesity.

14. The method according to claim 12, wherein the GLP-1 derivative is: 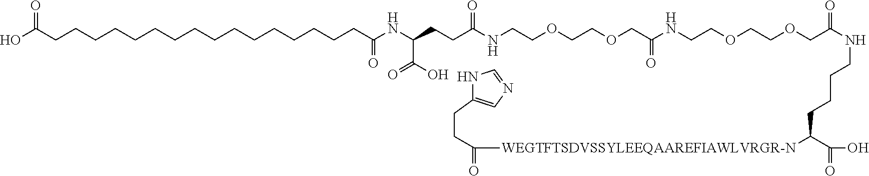
(Compound 1) or a pharmaceutically acceptable salt, amide, or ester thereof.

15. The method according to claim 14, wherein the subject is suffering from obesity.

16. The method according to claim 12, wherein the GLP-1 derivative is: 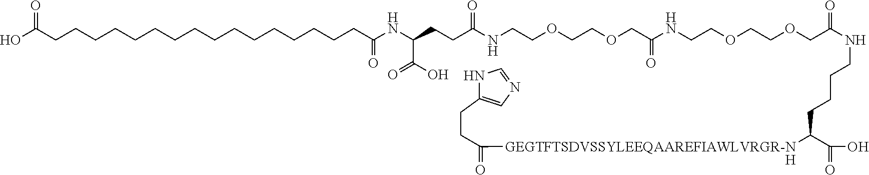
(Compound 2), or a pharmaceutically acceptable salt, amide, or ester thereof.

17. The method according to claim 16, wherein the subject is suffering from obesity.
